(19) United States
(12) Patent Application Publication
(10) Pub. No.: US 2015/0231109 A1
(43) Pub. Date: Aug. 20, 2015

(54) COMBINATIONS (CATECHINS AND METHOTREXATE) FOR USE IN THE TREATMENT OF MELANOMAS

(71) Applicant: UNIVERSIDAD DE MURCIA, Espinardo (ES)

(72) Inventor: Jose Neptuno Rodriguez-Lopez, Muricia (ES)

(21) Appl. No.: 14/628,151

(22) Filed: Feb. 20, 2015

Related U.S. Application Data
(63) Continuation-in-part of application No. PCT/EP2013/066934, filed on Aug. 13, 2013.

(30) Foreign Application Priority Data
Aug. 21, 2012 (GB) 1214877.1

(51) Int. Cl. A61K 31/353; G01N 33/50; A61K 31/519

(52) U.S. Cl. CPC A61K 31/353; A61K 31/519; G01N 33/5011; G01N 2333/90245

(57) ABSTRACT

The invention provides a method of treatment of melanoma comprising administering a tyrosinase expression enhancer, such as MTX, and a tyrosinase-activated prodrug, such as TMECG or TMCG, to an individual in need thereof. Also provided is a method of treating melanoma comprising administering a tyrosinase-activated prodrug and a compound for differentiating a stem-like tumor cell into a matured cell that is a tyrosinase producer to an individual in need thereof. Further provided is a method of treatment of melanoma comprising administering a tyrosinase expression enhancer and a tyrosinase-activated prodrug to an individual in need thereof, wherein the individual has a melanoma in which one or more of BRAF, NRAS, p53, GNAQ, EGFR, PDGFR, RAC or c-kit carries a mutation.

### RELATED APPLICATIONS AND INCORPORATION BY REFERENCE

This application is a continuation-in-part application of international patent application Serial No. PCT/EP2013/066934 filed 13 Aug. 2013, which published as PCT Publication No. WO 2014/029669 on 27 Feb. 2014, which claims benefit of GB patent application Serial No. GB 1214877.1 filed 21 Aug. 2012.

The foregoing applications, and all documents cited therein or during their prosecution (“appln cited documents”) and all documents cited or referenced in the appln cited documents, and all documents cited or referenced herein (“herein cited documents”), and all documents cited or referenced in herein cited documents, together with any manufacturer's instructions, descriptions, product specifications, and product sheets for any products mentioned herein or in any document incorporated by reference herein, are hereby incorporated herein by reference, and may be employed in the practice of the invention. More specifically, all referenced documents are incorporated by reference to the same extent as if each individual document was specifically and individually indicated to be incorporated by reference.

### FIELD OF THE INVENTION

This invention relates to compositions and methods for the treatment of melanoma and other cancer conditions.

### BACKGROUND TO THE INVENTION

A leading cause of therapeutic resistance in cancer is the combination of genetic and phenotypic heterogeneity within tumors. Molecularly targeted therapies may be bypassed by selection of genetically resistant cells, while reversible, microenvironment-driven, phenotypic heterogeneity may generate cells with stem cell-like properties that provide a pool of cells resistant to conventional chemotherapy (Visvader; Blagosklonny). During the past ten years, the incidence and annual mortality of melanoma has increased more rapidly than any other cancer and according to an American Cancer Society estimate, there will have been approximately 68,720 new cases of invasive melanoma diagnosed in 2009 in the United States, which resulted in approximately 8,650 deaths (American Cancer Society, 2009). The risk factors for developing melanoma are both environmental and genetic. The correlation of melanoma incidence with environmental exposure and biological traits points to multiple elements that may predispose an individual to melanoma (see Rhodes). Treatment options have remained remarkably static over the past 30 years (see Sullivan), although there have been some recent developments in the field, as noted below.

Although many patients with melanoma localized to the skin are cured by surgical excision, those with regional lymphatic or metastatic disease require radiation and chemotherapy (Tawbi). The most widely used chemotherapeutic agents are dacarbazine and its prodrug form temozolomide, both of which are used to treat metastatic melanomas. Presently dacarbazine is the only drug approved by the US Food and Drug Administration (FDA) for this indication. The response rate to dacarbazine is about 10 to 20% (see Serone). The use of temozolomide for the treatment of metastatic melanomas does not improve overall survival and progression-free survival when compared to treatment with dacarbazine (see Patel).

Currently, limited therapeutic options exist for patients with metastatic melanomas, and all standard combinations currently used in metastasis therapy have low efficacy and poor response rates. For instance, dacarbazine, has a response rate of about 10% and a median survival of 8-9 months. The other approved agent for advanced melanoma is high dose interleukin-2, which can induce dramatic complete and durable responses (Ascierto). However, only one patient in twenty derives lasting benefit. These data indicate the needed for alternative therapies for this disease and recent results indicated that combined therapies could became an attractive strategy to fight melanoma.

There is at present no evidence to indicate that combination chemotherapy, defined as any regimen containing a combination of one or more cytotoxic agent, is more effective than the marginal response rate by dacarbazine for patients with metastatic melanoma (see Chapman).

An increased understanding of melanocyte biology and melanoma pathogenesis has led to the development of targeted therapies which have the potential for major improvements in the care of patients with advanced melanoma. The most important breakthrough is the discovery that the mitogen-activated protein kinase (MAPK) pathway drives tumoregenesis. For example, metastatic melanoma containing V600E mutations in BRAF (a protein that activates the MAPK pathway), is a highly aggressive skin cancer with poor prognosis (Lopez-Bergami). This mutation is found in around 50% of melanomas. Targeting activated BRAF V600E with vemurafenib leads to dramatic and rapid tumor regression (Davies). However, the drawback of this treatment is the acquired resistance arising from mutations that bypass the requirement for activated BRAF V600E in MAPK signalling (Sosman; Villanueva). Additionally, there is currently no effective therapy for the 15-20% of melanomas with activated NRAS or other commonly known mutations.

Alternative FDA-approved therapies for disseminated melanomas include immune modulators such as IL-2 and anti-CTLA-4 (Ipilimumab), which are administered at high dose, and the B-Raf enzyme inhibitor Vemurafenib (marketed as Zelboraf). Other therapies for use in melanoma treatment include Tafinlar (dabrafenib) and Mekinist (trametinib), a MEK inhibitor. The therapeutic antibody Yervoy (ipilimumab) is also described for use in stimulating a patient's immune response. The vast majority of the responses to these regimens are partial and even good responses are often followed by relapses in which the recurring tumor has acquired substantial resistance to the therapy. Thus, the reported poor response of the combination of chemotherapy and immunomodulators, or using two or more anticancer drugs to treat metastatic melanomas in patients, underscores a need to develop new compounds for treatment of melanoma.

Some of the present inventors have previously described the use of catechin compounds, such as 3,4,5-trimethoxy-epicatechin-3-gallate (TMECG) and 3,4,5-trimethoxy-catechin-3-gallate (TMCG), to treat melanoma and other cancers (see WO 2009/081275). Such compounds are found to reduce dihydrofolate reductase (DHFR) activity. As a consequence of DHFR reduced activity, intracellular levels of tetrahydrofolate (THF) coenzymes decrease, resulting in inhibition of thymidine synthetase (TS) and depletion of nucleotides essential for DNA biosynthesis. Consequently, purine synthesis and DNA biosynthesis are inhibited. It has been shown that TMECG is a prodrug that is also a mild inhibitor of DHFR. The potent inhibition of DHFR is effected by the quinoine methide (QM) form of TMECG which is obtained by action of the tyrosinase enzyme on TMECG. The activation of TMECG occurs in a cell where tyrosinase is localized. Thus, TMECG and its related compounds may be regarded as anticancer prodrugs activated by specific enzyme catalysis.

Prodrugs are compounds that need to be transformed before exhibiting their pharmacological action and are often divided into two groups: (1) those designed to increase the bioavailability and/or improve the pharmacokinetics of antitumor agents and (2) those designed to deliver antitumor agents locally. Catechins such as TMECG have both these characteristics. TMECG is a prodrug which has good bioavailability in the blood. Upon activation by the melanocyte specific enzyme tyrosinase, TMECG is converted to a stable and biologically active QM from. Because the QM form is not bioavailable at plasma pH, and is only generated when TMECG is processed intracellularly in melanocytes, the QM form will accumulate at the site of conversion, thereby presenting an advantage over other drugs because it targets a specific cell type.

Therapies with TMECG would have good bioavailability and would also achieve a high local concentration of the drug. Chemoprevention is an ideal strategy for fighting melanoma. Many chemopreventive agents act through the induction of apoptosis as a mechanism for the suppression of carcinogenesis by eliminating genetically damaged cells, initiated cells or cells that have progressed to malignancy. The soft antifolate character of the prodrug (TMECG), its specific activation on melanoma cells, and the fact that antifolates are more active on fast-dividing cancer cells, make this compound ideal for the prevention and treatment of this skin pathology. The use of a TMECG prodrug is a potential strategy to overcome the limitations of chemotherapeutic agents that are non-selective for tumor cells, or indeed non-selective for melanoma cells.

As some of the present inventors have previously noted, the use of the catechin compounds is associated with an increase in the amount of folate receptor alpha (FRa) in the cell membrane of melanoma cells. The compounds are therefore useful in sensitising melanoma cells to cytotoxic FRa ligands. The antiproliferative effect of the catechin compounds is increased or potentiated by compounds which inhibit the methionine cycle. The antiproliferative effect of the catechin compounds is also increased or potentiated by compounds which reduce or inhibit the level of dihydrotestosterone (DHT) in cells. Additionally, thymidylate synthase inhibitors potentiate the effect of the catechin compounds, particularly in male patients.

Citation or identification of any document in this application is not an admission that such document is available as prior art to the present invention.

### SUMMARY OF THE INVENTION

The present inventors have found that the effects of a catechin compound in a method of treatment may be potentiated by the administration of a compound that increases the conversion of the catechin compound to its more active quinoine methide (QM) form. The conversion of a catechin compound to the QM form is an oxidation step, mediated by an enzyme, such as tyrosinase. Increasing the level of the enzyme, such as tyrosinase, present in a cell increases the amount of QM form produced.

The present inventors have recognised that the activation of tyrosinase expression may be used advantageously to increase the conversion of a tyrosinase-activated prodrug to its active form.

The strategy of enhancing the conversion of a prodrug, such as a catechin compound, to its active form in melanoma cells represents an alternative and attractive strategy to the chemotherapeutic treatments described in the prior art that are non-selective for melanoma. In a general aspect the present invention provides a method of treatment, the method which may comprise the step of administering a compound which increases the level of tyrosinase in a cell (a “tyrosinase expression enhancer”) in combination with a tyrosinase-activated prodrug compound. In one embodiment, the tyrosinase-activated prodrug compound is a catechin compound.

The present inventors have established that a compound which increases the level of tyrosinase in a cell may be used together with a catechin compound to provide an enhanced treatment of tumors, and particularly melanomas.

The combination of a tyrosinase expression enhancer and a tyrosinase-activated prodrug is highly effective in vitro and in vivo and has several key advantages compared to more conventional strategies. The effectiveness of the therapy is strictly dependent on processing of the pro-drug by TYR, a melanocyte-specific gene, thereby avoiding damage to other cell types which is a major disadvantage of conventional chemotherapies. By inducing dTTP-depletion through targeting an essential enzyme, the combination therapy is effective in melanoma cells irrespective of their BRAF or MEK status, and is not susceptible to resistance arising from genetic heterogeneity within the MAPK pathway, the major cause of resistance to anti-BRAF therapies. The pro-apoptotic effect of dTTP depletion in response to the combination is independent of p53 status. Thus, the combination overcomes many of the genetic and phenotypic heterogeneity issues that are major barriers to current anti-melanoma therapy. Moreover, where the tyrosinase expression enhancer is capable of up-regulating MITF, the tyrosinase expression enhancer potentially depletes the pool of invasive melanoma cells that drive metastasis formation.

MITF is a key transcriptional regulator, therefore up-regulating or activating MITF has the dual benefit of increasing TYR expression and driving cellular differentiation.

In a first aspect of the invention there is provided a composition for use in a method of treatment, the composition which may comprise a compound which increases the level of tyrosinase in a cell and a catechin compound.

In a second aspect of the invention there is provided a composition for use in the treatment of cancer, such as melanoma, the composition which may comprise a compound which increases the level of tyrosinase in a cell and a catechin compound.

In another aspect of the invention there is provided a pharmaceutical composition which may comprise a compound which increases the level of tyrosinase in a cell and a catechin compound. Also provided is a kit which may comprise a compound which increases the level of tyrosinase in a cell and a catechin compound.

In a further aspect there is provided the use of compound which increases the level of tyrosinase in a cell and/or a catechin compound in the manufacture of a medicament for the treatment of a cancer, such as melanoma, wherein the medicament may comprise the compound which increases the level of tyrosinase in a cell and the catechin compound.

In one aspect there is provided a method of treatment, the method which may comprise administering to a patient in need thereof a compound which increases the level of tyrosinase in a cell and a catechin compound. The method of treatment may be the treatment of a cancer, such as melanoma.

In one aspect the present invention provides a method of treatment, the method which may comprise administering to a subject a compound which increases the level of tyrosinase in a cell, wherein the subject has undergone treatment with a catechin compound. In a related aspect the present invention provides a method of treatment, the method which may comprise administering to a subject a catechin compound, wherein the subject has undergone treatment with a compound which increases the level of tyrosinase in a cell. In these aspects the method of treatment is of a cancer, such as melanoma, for example invasive melanomas. The subject may be a human subject.

In a further aspect of the invention there is provided a method of treatment, the method which may comprise administering to a subject who has undergone treatment with an inhibitor of a BRAF mutant a compound that increases the level of tyrosinase in a cell, together with a catechin compound. In this aspect, the method of treatment is of a cancer, such as melanoma, for example invasive melanomas. The subject may be a human subject. In related aspects, the subject may be a subject who has undergone treatment with an inhibitor of a NRAS, p53, GNAQ, EGFR, PDGFR, RAC or c-kit mutant.

In another aspect of the invention there is provided a method of treatment which leads to the differentiation of stem-like tumor cell, such as melanoma stem-like cell. The method may comprise administering to a subject a compound which differentiates a stem-like tumor cell into a matured cell that is a tyrosinase producer. The invention also provides treatment of the differentiated cell with a catechin compound.

The present invention also provides methods for identifying compounds which increase tyrosinase levels in a cell.

The compound which increases tyrosinase levels in a cell may be a compound that acts to increase MITF levels in a cell. The compound which increases tyrosinase levels in a cell may be methotrexate (MTX)

The compound which induces differentiation of stem-cell like melanomas may be MTX.

The catechin compound may be TMECG or TMCG.

The melanoma includes those melanomas containing mutations in the MAPKinase pathway and/or the melanocyte differentiation pathway. The mutations include mutations in one or more of BRAF, NRAS, p53, GNAQ, EGFR, PDGFR, RAC and c-kit.

Accordingly, it is an object of the invention not to encompass within the invention any previously known product, process of making the product, or method of using the product such that Applicants reserve the right and hereby disclose a disclaimer of any previously known product, process, or method. It is further noted that the invention does not intend to encompass within the scope of the invention any product, process, or making of the product or method of using the product, which does not meet the written description and enablement requirements of the USPTO (35 U.S.C. §112, first paragraph) or the EPO (Article 83 of the EPC), such that Applicants reserve the right and hereby disclose a disclaimer of any previously described product, process of making the product, or method of using the product. It may be advantageous in the practice of the invention to be in compliance with Art. 53(c) EPC and Rule 28(b) and (c) EPC. Nothing herein is to be construed as a promise.

It is noted that in this disclosure and particularly in the claims and/or paragraphs, terms such as “comprises”, “comprised”, “comprising” and the like can have the meaning attributed to it in U.S. Patent law; e.g., they can mean “includes”, “included”, “including”, and the like; and that terms such as “consisting essentially of” and “consists essentially of” have the meaning ascribed to them in U.S. Patent law, e.g., they allow for elements not explicitly recited, but exclude elements that are found in the prior art or that affect a basic or novel characteristic of the invention.

These and other embodiments are disclosed or are obvious from and encompassed by, the following Detailed Description.

### DESCRIPTION OF THE FIGURES

The following detailed description, given by way of example, but not intended to limit the invention solely to the specific embodiments described, may best be understood in conjunction with the accompanying drawings.

FIGS. 1A-J. Methotrexate up-regulates the expression of MITF. A, The MITF rheostat, and the two-step strategy for melanoma therapy: Drug A induces MITF, driving cells to be sensitive to Drug B. B, MTX (1 μM) increases MITF mRNA determined using quantitative and conventional RT-PCR from indicated cell lines. Asterisks denote statistically significant differences (P<0.05). C, MTX (1 μM) induces MITF protein assayed by western blotting (WB) (*P<0.05). D, Immunofluorescence of control or MTX-treated (3 h) 501 MEL cells using anti-MITF and DAPI. E, Matrigel assay of control and MTX-treated SKMEL-28 cells (48 h, 1 μM) and IGR39 (72 h, 1 μM) cells. Asterisks denote statistically significant differences (*p<0.05). Scale bar refers to all panels. F, Chromatin immunoprecipitation on TYR and Pmel17 genes of control and MTX-treated (4 h, 1 μM) SK-MEL-28 cells with qRT-PCR after immunoprecipitation with IgG, or MITF and HDAC3 antibodies. Error bars indicate standard deviations of triplicates; experiment reproduced four times with similar results. G, Scanning electron micrographs of control and MTX-treated (1 μM, 24 h) SK-MEL-28 cells. H, Semiquantitative RT PCR of TYR, TYRP1, Pmel17, MART1, and Rab27a mRNA. SK-MEL-28 and siMITF-SK-MEL-28 cells were treated for 5 h with 1 μM MTX. mRNA levels are presented relative to β-actin mRNA and compared to their expression levels in untreated cells (1-fold). Induction of all genes by MTX was statistically significant (P<0.05), but not in siMITF-SK-MEL-28 cells. WB indicates efficiency of MITF knockdown using MITF-specific stealth RNA oligonucleotide (siMITF) compared to control (siCN). I, WB for TYR in indicated cell lines (upper panels) following MTX treatment for indicated times; and immunofluorescence for TYR (green) and the melanosome stage II marker, HMB45 (red) (lower panels), in SKMEL-28 cells before (control) and after MTX (1 μM, 3 h) treatment. DAPI is represented in blue. J, WB of MART1 protein levels in melanoma cell lines (*P<0.05) following MTX treatment (1 μM).

FIGS. 2A-N. MTX and TMECG combination therapy induces apoptosis via dTTP depletion and DNA-damage. A, Intracellular accumulation of TMECG-QM in SK-MEL-28 treated with TMECG or MTX/TMECG for 24 h. B, Proliferation assays performed of control or MTX/TMECG-treated (10 μM/1 μM) SK-MEL-28 cells. C, Quantification of effects of MTX/TMECG treatment on SK-MEL-28 cells (*P<0.05 with respect to TMECG-treated cells). For time course assay MTX (1 μM) and TMECG (10 μM) were used. D, dNTP quantification in melanoma cells subjected to indicated treatments (*P<0.05). E, SK-MEL-28 cells treated with MTX/TMECG for the indicated times were examined by immunofluorescence for γH2AX foci (red) and DAPI (blue) (left panels), or by WB (right panels). β-actin served as a protein loading control. F, apoptosis determination at different MTX/TMECG combinations in SK-MEL-28 cells after 4 days of treatment. Data were obtained in triplicate in two independent experiments. Differences in apoptosis in MTX/TMECG treated cells were significant with respect to individual treatments for each drug concentration (p<0.05). G, MTT assay indicating effects of MTX/TMECG (1 μM/10 μM) and PLX-4720 (1 μM) treatment on low passage patient-derived melanoma cells bearing indicated MEK and BRAF mutations. Cells were treated with vehicle only (•), PLX-4720 ( ), or with a combination of MTX+TMECG (Δ). Note the number of cells at the start of the experiment was at the limit of detection. H, effects of MTX/TMECG (1 μM/10 μM) on cell number of indicated cell lines. Error bars show mean±SD. I, dTTP quantification in melanoma cells subjected to indicated treatments. *p<0.05 with respect to untreated controls; **p=0.001; ***Not statistically significant with respect to the untreated controls. J, SK-MEL-28 cells treated with MTX (1 μM) and/or TMECG (10 μM), or X-rays (1 Gy) were examined by immunofluorescence for γH2AX foci (red) and DAPI (blue). Scale bar=7 μm and refers to all panels. K, quantification of γH2AX foci in SK-MEL-28 cells treated with MTX (1 μM) and/or TMECG (10 μM), or X-rays. Histograms represent the positive γH2AX foci cells and γH2AX foci/nucleus in positive γH2AX foci cells (*p<0.01 when compared with untreated cells or those subjected to MTX and TMECG individuals treatments). L, Detection of γH2AX by WB in SK-MEL-28 cells treated with MTX and/or TMECG. β-actin served as a protein loading control. M, Comet assay of SK-MEL-28 treated with vehicle or MTX/TMECG (1 μM/10 μM) for 48 hr. Hydrogen peroxide was used as a positive control (data not shown). Scale bar refers to both panels. N, Cell cycle profile determined by flow cytometry of SK-MEL-28 cells treated with a sublethal dose of MTX/TMECG. Error bars show mean±SD.

FIGS. 3A-F. MTX/TMECG treatment induces E2F and p73 but not p53. MTX 1 μM and TMECG 10 μM were used (for all changes *P<0.05 with respect to individual treatments in all expts). A, Indicated combinations of drugs were added to melanoma cell lines (p53 status indicated) and apoptosis determined after 3 days. p53 was silenced in G361 cells as indicated and shown in inset WB. B, qRT-PCR analysis of TAp73, p53, and Apaf1 (left panel) in SKMEL-28 treated with MTX and/or TMECG. Apaf1 protein levels are shown (right panel). β-actin was used as a protein loading control. mRNA levels are presented relative to β-actin mRNA. C, p73 protein levels were evaluated by WB over time following indicated treatments. β-actin was used as loading control. D, WB of p-Chk1, p-Chk2, and E2F1 after MTX/TMECG treatment. E, Immunoprecipitation of E2F1 from control or MTX/TMECG-treated SK-MEL-28 cells and WB of immunoprecipitates using indicated antibodies. β-actin served as a protein loading control. F, Apoptosis assays of cells transfected with siRNA and treated with vehicle or MTX/TMECG.

FIGS. 4A-N. MTX and TMECG combination therapy is effective in vivo. A, A375 melanoma cells were included in a human reconstructed skin model and were treated with MTX (1 μM) and/or TMECG (10 μM) for 14 days. B, B16/F10 melanoma cells were injected subcutaneously into C57/B16 mice. Images show tumor size 17 days post-injection in control and treated mice. The time-dependent evolution of tumors and the mean tumor area after 21 days of treatment are shown. *P<0.05; **P<0.005; NS=not statistically significant. C, Luciferase imaging of control and MTX/TMECG-treated mice 12 days post-tumor cell injection. Beetle luciferin (120 mg/kg of mouse) was injected intraperitoneally. The values (means±s.d.) are representative of three independent experiments. *P=0.007; **P=0.002. D, Bioluminescent imaging of livers at 14 days post-intrasplenic injection of B16-F10-luc-G5 cells from untreated and MTX/TMECG-treated mice are shown. E, Quantification of macrometastases (0-10, 10-25 or >25) after treatment with vehicle (control), MTX (1 mg/kg/day), and/or TMECG (50 mg/kg/day). F, Photomicrograph of H&E-stained, 4 μm formalin-fixed, paraffin-embedded liver sections from control (DMSO) and MTX/TMECG-treated mice (1 and 50 mg/kg/day, respectively) (5× magnification). G, MTX activation of MITF and tyrosinase activates the melanoma-specific antifolate activity of TMECG, leading to depletion of cellular dTTP and apoptosis. H, Sections stained with hematoxylin and eosin show the effect of MTX/TMECG on B16/F10 primary splenic tumors. Xenograft tumors treated with DMSO (vehicle) or MTX/TMECG (1 mg/kg/day and 50 mg/kg/day, respectively) over 14-days. Vehicle-treated tumors showed no discernible necrosis (N), while MTX/TMECG-treated tumors showed hemorrhagic (H) necrosis with obvious dividing line between viable (T) and necrotic tissues. Representative images taken from two independent experiments (n=5 for each experiment). Scale bar=200 μm and is applicable to both panels. I, B16/F10 melanoma cells, expressing a luciferase reporter, were injected subcutaneously to mice. Mice were divided in two groups (n=7) and treated with vehicle (DMSO) or MTX/TMECG (1 mg/kg/day and 10 mg/kg/day, respectively) over 21-days. Then, tumors were pooled and dissociated. B16/F10 cells extracted from tumors were examined for their sensitivity to the MTX/TMECG combination (1 μM and 10 μM, respectively) using quantification of the luminescence signal (left panel). J, The histogram represents the number of B16/F10-luc2 cells remaining after 3 days of MTX/TMECG treatment with respect to vehicle treated controls (100%). Mean±SD was calculated in triplicate (NS, not statistically significant). K, The morphology of tumor dissociated B16/F10-luc2 cells before and after of MTX/TMECG treatment. Scale bar refers to all panels. L, Histograms represent the number of copies of TYR mRNA for every 1×103 copies of β-actin±SD of three independent experiments. Mice were treated with vehicle, MTX (1 mg/kg/day), and/or TMECG (50 mg/kg/day). Asterisks show statistically significant differences when compared with untreated controls (vehicle) (*p=0.005; **p=0.001). Livers from non-inoculated mice (NT) were used as a control. M, Toxicological assays of the effect of MTX and/or TMECG on skin melanocyte integrity. MTX/TMECG treatment (20 days; 1 mg/kg/day and 50 mg/kg/day, respectively) did not influence number and morphology of mouse skin melanocytes. N, Microscopic analysis (40× magnification) H&E stain of mouse retina and retinal pigmented epithelium (RPE) and MITF immunostaining (Left Panel), or Iridal melanocytes (IPE) (right panel) indicating no obvious differences following 20 days MTX/TMECG treatment. Scale bar refers to all panels.

FIGS. 5A-D. The effects of MTX and/or TMECG on the growth of melanoma cell lines. A, Viability was determined by the MTT assay. Cells were treated with vehicle only (•), 10 μM TMECG ( ), 1 μM MTX (▴) or with a combination of 1 μM MTX+10 μM TMECG (▾). Table outlines p53, BRAF, NRAS, and PTEN status with specific amino acid substitutions. NRAS showed a wild-type (WT) phenotype in all melanoma cell lines tested. B, MTX/TMECG synergy test for melanoma and non-melanoma cancer cells in which drugs were combined in 6×6 matrices where the concentration of one drug was increased along each axis. The lowest concentration was 0 and the highest concentration was chosen close to the IC₅₀ for each drug in each assayed cell line. Apoptosis was determined after 4 days of treatment. Data were obtained in triplicate in two independent experiments. C, Apoptosis assays of SK-MEL-28 cells transfected with control or MITF-specific siRNA and treated with vehicle or MTX/TMECG (1 μM/10 μM). Insert WB indicates efficacy of siRNA. Data are presented as percentage apoptosis compared to MTX/TMECG-treated siCN cells (100%) and represent the mean±SD from three independent experiments. *p<0.05 when compared with siCN-treated cells. D, Apoptosis assays of SK-MEL-28 cells transfected with control or TYR-specific siRNA and treated with vehicle or MTX/TMECG (1 μM/10 μM). Insert WB indicates efficacy of siRNA. Data are presented as percentage apoptosis compared to MTX/TMECG-treated siCN cells (100%) and represent the mean±SD from three independent experiments. *p<0.05 when compared with siCN-treated cells.

FIGS. 6A-C. MTX/TMECG modulates the posttranslational state of E2F1. A, Schematic representation of the E2F1 protein. Residues susceptible to methylation (K185), acetylation (K117, K120, and K125), and phosphorylation (S31 and S364) are shown. B, MALDI-TOF mass spectra of phosphorylated and nonphosphorylated peptides (at Ser31 and Ser364) in E2F1-trypsin digested samples. Peptides were analysed in untreated SK-MEL-28 cells (Control) or treated for 10 h with 1 μM MTX+10 μM TMECG (MTX/TMECG). The molecular mass of phosphorylated peptide is 80 Da larger than that of the nonphosphorylated peptide under the defined MS settings. C, Proposed mechanism for the regulation of E2F1. E2F1 is regulated by several posttranslational modifications, including methylation (Me), acetylation (Ac) and phosphorylation (P). The effects of MTX (red dashed line) and MTX/TMECG (green dashed line) on E2F1 status are shown. E2F1 is reversibly methylated by the enzymatic actions of lysine-specific demethylase 1 (LSD1) and histone methyltransferase (Set9).

FIGS. 7A-C. A, Luciferase imaging of vehicle (control), MTX, and/or TMECG-treated mice 12 days post-tumor cell injection. Firefly luciferin (120 mg/kg of mouse) was injected intraperitoneally. The values are representative of three independent experiments. The right panel shows time dependent effects of treatment on the number of tumor cells in spleen-induced tumors. The number of cells was estimated by extrapolation of the experimental luciferase signal to calibration curves. A straight line with a good linear correlation coefficient (0.998) was obtained by plotting luciferase signal vs number of B16/F10-luc2 cells. Treatment of mice (n=6) started 24 hr after tumor induction (Day 1). For all experiments: *p<0.05 with respect to control mice; #p<0.05 with respect to individual MTX or TMECG treatments. B, Left panel, MITF protein levels were evaluated by WB in primary spleen tumors. Spleens (n=6) were obtained from mice after 14 days post-intrasplenic injection of B16/F10-luc2 cells. Mice were treated with vehicle or 1 mg/kg/day MTX. Histograms bars represent MITF levels relative to β-actin and compared to their levels in untreated mice (1-fold). *p<0.05. Right panel, vehicle- and MTX-treated (1 mg/kg/day; 15 days) B16/F10-subcutaneous tumors (n=5) were dissociated and cells assayed for MITF expression by WB (*p<0.05 with respect to controls). C, Dissociated tumor cells isolated from B16/F10 subcutaneous tumors (as in C) were assayed by confocal microscopy for MITF (red) MART1 (green) and DAPI (blue). Representative confocal microscopy images immunostained with two different MITF antibodies are shown. Error bars in the entire figure show mean±SD.

FIGS. 8A-C. A, Mean plasma MTX concentration at different times after intra-peritoneal injection of male C57BL/6 mice with either MTX alone or co-injected with TMECG. B, Mean plasma TMECG concentration at different times after intra-peritoneal injection of male C57BL/6 mice with either TMECG alone or together with MTX. C, Graphs representing the median of the mouse body weight±SD following injection with MTX and/or TMECG. Differences between untreated and TMECG-treated groups were not significant during the whole experiment. On the eleventh day of the treatments, differences in body weight of 10 mg/kg/day MTX-treated mice versus untreated or TMECG-treated groups were statistically significant (p<0.05) and, to avoid unnecessary suffering of the animals, the survivors of the 10 mg/kg/day MTX-treated group were sacrificed at this time. Differences between the untreated group and those treated with a combination of MTX and TMECG (at doses 1 mg/kg/day and 50 mg/kg/day, respectively) were not significant during the whole experiment. See also Table 3 for pharmacokinetic parameters such as maximum plasma concentration (Cₘₐₓ), time of maximum concentration (Tₘₐₓ), AUC, or elimination half-life (t_{1/2}).

### DETAILED DESCRIPTION OF THE INVENTION

Some of the present inventors have previously described the use of TMECG to treat tumors, such as melanoma and breast cancer. The mechanism of action involves the conversion of the TMECG to its quinone methide (QM) form by, for example, tyrosinase (TYR).

Some of the present inventors have shown that the antiproliferative action of TMECG is mitigated if TYR expression is silenced, for example using siRNA. It has also been shown that the delivery of TYR into cancer cells together with TMECG enhances the antiproliferative effect of TMECG, for example inducing cell growth inhibition and apoptosis.

The present inventors have now found that the effects of TMECG may be enhanced if the expression levels of TYR are increased within the cell, thereby to increase the amount of quinone methide formed. In their previous work, the inventors have increased cellular TYR levels by delivering TYR into a colorectal cancer cell as a conjugate with folate (FOL-TYR).

Now, the present inventors provide methods and compositions which may comprise active compounds that increase cellular TYR expression levels, either directly or indirectly (e.g. via MITF upregulation). Such methods do not require the delivery of a TYR conjugate into the cell. Thus, the active compound is not TYR itself. Rather, the inventors have identified a class of compounds that is capable of enhancing TYR expression. The compounds may be used to increase TYR expression in melanoma cells. Thus, the compounds may be referred to as TYR expression enhancers.

The inventors have found that the TYR expression enhancer acts to increase the conversion of TMECG to the quinone methide form, which in turn provides increased inhibition of DHFR and lower levels of dTTP, thereby resulting in an improved anti-proliferative effect.

It is clear that this strategy has a more general application, and a TYR expression enhancer may be used increase the conversion of a TYR-activated prodrug to its active form.

Advantageously, the inventors have found that a TYR expression enhancer having antiproliferative activity may be used to enhance the overall antiprolfilerative effects. As demonstrated herein, methotrexate (MTX) acts to increase tyrosinase levels. MTX is also known as an anticancer compound and it use is associated with reduced cancer cell proliferation. In particular MTX reduces DHF levels within a cancer cell.

Although methotrexate (MTX) is an efficient drug for several types of cancer, it is not active against melanoma (Kufe et al., 1980). Its use by the present inventors to promote phenotype-switching, to render cancer cells sensitive to prodrug therapy, is therefore a useful development of the work on MTX, and could improve current therapeutic approaches.

By way of example, the present inventors have shown that an active agent, such as MTX, may be used to increase TYR expression, thereby increasing the conversion of a catechin to its quinone methide form. The inventors have undertaken HPLC analyses of MTX, TMECG-QM, and DHF in whole cell extracts of SK-MEL-28 melanoma cells that were subjected to MTX (1 μM) and TMECG (10 μM) individual and combined treatments (see Table 1). The results show that the levels of the quinone methide form may be increased by more than 5-fold in the presence of methotrexate and TMECG (compared to the treatment with TMECG alone). The results show that DHF levels are retained at a low level in a combination treatment with methotrexate and TMECG.

The inventors have found that the combination of agents provides a highly beneficial treatment result. As shown herein, the combination of methotrexate with the catechin TMECG provides an improved treatment regime.

As will be appreciated, TMECG may be replaced with an alternative catechin compound, such as TMCG, or an alternative tyrosinase-activated prodrug.

It is known that TYR expression is influenced by MITF, the Microphthalmia-associated transcription factor. Elevated MITF levels increase TYR expression. The inventors have now established that the relationship between MITF expression and TYR expression may be used advantageously to harness the effects of MTX to increase the conversion of catechin to its active form.

First, by up-regulating MITF, MTX potentially depletes the pool of MITF-negative stem-like cells that have been shown to drive tumor initiation in synegenic mouse models and which may represent a pool of slow-proliferating, cells resistant to conventional chemotherapy (Cheli et al. 2011); second, processing of the TMECG pro-drug by TYR is melanocyte-specific, thereby avoiding damage to other cell types; third, by targeting the essential enzyme DHFR and inducing dTTP-depletion, MTX/TMECG combination therapy acts independent of BRAF, NRAS or p53 status and should not be susceptible to resistance arising from bypassing the molecular target, as is the case with BRAF inhibitors. Importantly, MTX is in widespread clinical use for a variety of steroid-recalcitrant inflammatory diseases, and our preliminary observations indicate that TMECG is not toxic in vivo. As such MTX/TMECG combination therapy has potential for rapid application in a human setting.

Thus, the combination of a tyrosinase expression enhancer (such as MTX) and a tyrosinase-activated prodrug (such as TMECG) overcomes many of the genetic and phenotypic heterogeneity issues that are major barriers to current anti-melanoma therapy. Among the residual population of tumor cells in combination-treated mice, resistance was not detected.

In some commentaries it has been noted that increased MITF expression may be associated with melanocyte survival. It is said that UV radiation causes increased expression of transcription factor p53 in keratinocytes, and p53 causes these cells to produce melanocyte-stimulating hormone (MSH), which binds to melanocortin 1 receptors (MC1R) on melanocytes. Ligand-binding at MC1R receptors activates adenylate cyclases, which produce cAMP, which activates CREB, which promotes MITF expression. The targets of MITF include p16 (a CDK inhibitor) and Bcl2, a gene essential to melanocyte survival. It is postulated the impedance of this pathway, for example upstream of MITF, may be a suitable therapeutic strategy. In contrast, the present inventors have shown that increased expression of MITF is a strategy that may be used to increase tyrosinase, which is used to generate an active drug form for the treatment of cancers such as melanoma.

Additionally, increased expression of MITF also drives the differentiation of stem-like cells that contribute to tumorigenesis and invasiveness of the disease. Once differentiated, the previously unsensitized tumor-replenishing stem-like cell population should become susceptible to the active drug. Methods of the invention therefore extend to the use of compounds for the differentiation of stem-like cells.

Described herein are assays suitable for testing whether a compound has the ability to modulate, such as increase, TYR expression. Also described herein are assays suitable for testing whether a compound has the ability to modulate, such as increase, MITF expression. Changes in MITF expression levels may be indicative of a change in differentiation.

The methods and compositions described herein call for a compound which increases the level of tyrosinase in a cell. As noted herein, the compound which increases tyrosinase is not tyrosinase itself, but a compound that increases tyrosinase expression within the cell.

MTX (methotrexate) is a compound for use as tyrosinase expression enhancer. Forskolin is an alternative compound for use as tyrosinase expression enhancer. However, the use of compounds such as MTX is preferred owing to the antifolate activity of MTX, as discussed below. Moreover, the use of Forskolin is also associated with systemic toxicity.

Other tyrosinase expression enhancers that are contemplated for use in the present invention include U0126, PD0325901, and PLX4720 (as described by Boni et al.).

Described herein are suitable screening methods for identifying whether a compound has the ability to increase tyrosinase expression. Such methods may include monitoring changes in the conversion rate of a catechin compound to its QM form in a cell-based assay in response to a test compound. Such compounds may also be screened for their ability to increase the conversion of other TYR-activated prodrugs.

A compound which increases the level of tyrosinase in a cell may do so indirectly by increasing MITF levels in a cell. For example, the compound may increase both MITF mRNA and protein levels in either mouse or human cell lines. Increased MITF levels are associated with increased mRNA expression of the MITF differentiation targets TYR, thereby increasing levels of tyrosinase in the cell.

As described herein a compound may be screened for its ability to increase MITF levels in a cell. A compound having this ability may then be screened for its ability to increase tyrosinase levels in a cell.

It was observed that the tyrosinase expression enhancer MTX increases MITF expression, and consequently the expression of multiple melanosomal components. This may provide an explanation for the fact that melanomas, compared to epithelial cells, are highly resistant to the effects of MTX alone. Accumulating evidence indicates that melanosomes, whose biogenesis is promoted by MITF, contribute to the refractory properties of melanoma cells by sequestering cytotoxic drugs and increasing melanosome-mediated drug export. Moreover, folate receptor α (FRα)-mediated endocytotic transport of MTX facilitates melanosomal drug sequestration and cellular export in melanoma cells, thereby reducing the accumulation of MTX in intracellular compartments (Sanchez-del-Campo et al., 2009b). Thus MTX-driven up-regulation of MITF and consequent increased melanosome biogenesis may promote MTX resistance. In this respect, the combination of a tyrosinase-activated prodrug in combination with a tyrosinase expression enhancer bypasses this barrier to tyrosinase expression enhancer monotherapy.

Although MTX up-regulates MITF mRNA and protein expression, how MTX activates the MITF promoter is not fully understood, though preliminary results (not shown) indicate that MTX up-regulates expression of Sox10, a known regulator of MITF expression (Lee et al., 2000). MTX plays additional roles including inducing E2F1 demethylation and depletion of DHF pools.

Thus, the tyrosinase expression enhancer may be a compound that has one or more activities selected from the group consisting of up-regulation of Sox10 expression; induction of E2F1 demethylation; and depletion of DHF.

In one embodiment, the tyrosinase expression enhancer is an antifolate compound. Thus, the compound is capable of inhibiting the activity of dihydrofolate reductase (DHFR). Compounds having such an activity are particularly useful, as they can enhance the antifolate properties of the catechin compound. Thus, the use of a tyrosinase expression enhancer compound that is an antifolate compound provides, together with the catechin compound, an enhanced antifolate effect. In one embodiment, the antifolate compound is MTX.

Specifically, MTX acts to deplete levels of DHF within a cell, thereby reducing the amount of THF that may be produced by DHFR. In turn, this reduces the amount of deoxythymidine triphosphate (dTTP) produced. Thus, in one embodiment, the antifolate compound is a compound that decreases DHF levels in a cell, such as a cancer cell.

It is noted herein that MTX, used alone, may cause dTTP levels in a melanoma cell to increase. This is due to the effect of MTX acting to increase MITF levels. DHFR is a target gene for MITF, therefore increased MITF levels may be associated with increased dTTP levels. However, MTX is capable of reducing DHF levels, as shown by the present inventors when analysis whole cell extracts of SK-Mel-28 cells (see Table 1).

Thus, alternative antifolate compounds to MTX may be useful as tyrosinase expression enhancers in the present invention. For example, aminopterine, pemetrexed, raltitrexed or prelatrexate may be used as alternatives to MTX.

Tyrosinase expression enhancers additionally having an antifolate effect may be identified using the screening methods described herein. Here, for example, the ability of a compound to alter DHFR activity may be gauged by determining the amount of substrate DHF present in whole cell extracts of cancer cells treated with and without the tyrosinase expression enhancer.

In one embodiment, the tyrosinase expression enhancer is a compound that reduces the activity of one or more proteins in the MAP kinase pathway. Thus, the tyrosinase expression enhancer may be a MEK kinase inhibitor, such as U0126 or PD0325901. Additionally or alternatively, the tyrosinase expression enhancer may be a BRAF inhibitor, including those inhibitors of mutant BRAF, such as BRAF V600E. An example of a BRAF V600E inhibitor for use in the present invention includes PLX4720. As noted above in relation to MTX, the use of a compound having a tyrosinase expression enhancing activity and an anticancer effect may provide a greatly enhanced overall treatment strategy for those tumors treated with a tyrosinase-activated prodrug.

A tyrosinase expression enhancer may be used to increase the amount of tyrosinase present in a cell, such as a cancer cell. An increase in tyrosinase cell levels may be used to increase the conversion of a tyrosinase-activated prodrug to its active form.

Tyrosinase-activated prodrugs are known in the art. As described herein, catechin compounds are converted by tyrosinase to a more active quinone methide (QM) form. Tyrosinase-activated prodrugs are also used in Melanocyte-directed enzyme prodrug therapy (MDEPT), as described by Jordan et al. (Jordan et al. 1999; Jordan et al. 2001). Examples include prodrugs derived from 6-aminodopamine and 4-aminophenol (see Knaggs et al.).

In one embodiment, the tyrosinase-activated prodrug is for treatment of cancer. In a preferred embodiment, the tyrosinase-activated prodrug is for treatment of melanoma.

Thus, the activation of tyrosinase activity, for example through MITF activation, is an attractive approach to increasing the effectiveness of an anticancer drug.

It is preferred that the TYR-activated prodrug is a catechin compound, as described below.

An aspect of the invention provides a method of treating melanoma or other cancer which may comprise administering to an individual in need thereof a therapeutically effective amount of a catechin compound. The compound may be a compound (XI), which is converted to a compound of formula (X) by tyrosinase.

Related aspects provide a compound of formula (XI) for use in the treatment of melanoma or other cancer, and the use of a compound of formula (XI) in the manufacture of a medicament for use in the treatment of melanoma or other cancer.

In one embodiment, catechin compound is TMECG or TMCG

In some embodiments, the catechin compound is 3,4,5-trimethoxy-epicatechin-3-gallate (TMECG).

3,4,5-trimethoxy-epicatechin-3-gallate has the formula (I) below. The atom numbering is shown.

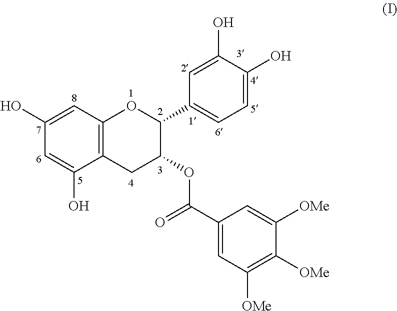 [SMILES: COc1cc(C(=O)O[C@@H]2Cc3c(O)cc(O)cc3O[C@@H]2c2ccc(O)c(O)c2)cc(OC)c1OC]

3,4,5-trimethoxy-epicatechin-3-gallate is activated within melanoma cells to a quinone methide (QM) metabolite having a deprotonated form at neutral pH which is shown in formulae (II) and (III) below.

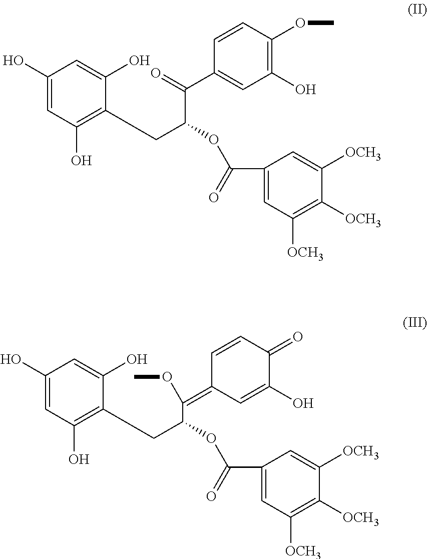

In other embodiments, the catechin compound is 3,4,5-trimethoxy-catechin-3-gallate:

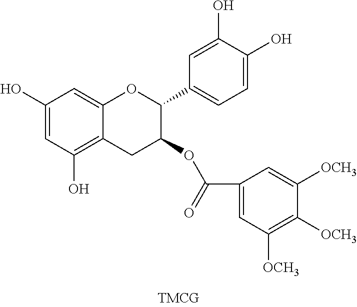 [SMILES: COc1cc(C(=O)O[C@H]2Cc3c(O)cc(O)cc3O[C@@H]2c2ccc(O)c(O)c2)cc(OC)c1OC]

Suitable for use in the methods described herein is a compound of formula (XI):

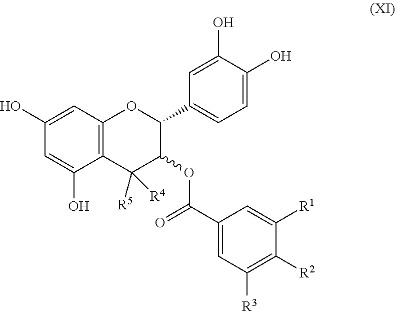

wherein:
each —R¹, —R² and —R³ is independently -Q¹, —OH or —H, where at least one of —R¹, —R² and —R³ is not —H or —OH;
each —R⁴ and —R⁵ is independently -Q² or —H;
each -Q¹ is independently selected from:
   —F, —Cl,
   —R^{A},
   —OR^{A},
   —SH, —SR^{A},
where each —R^{A} is independently selected from methyl and ethyl, which may substituted by one or more fluoro or chloro groups;
each -Q² is selected from:
   —F, —Cl,
   —R^{B},
   —OR^{B},
   —SH, —SR^{B},
where each —R^{B} is independently selected from methyl and ethyl, which may substituted by one or more fluoro or chloro groups or an isomer, salt, solvate or prodrug thereof.

In some embodiments, when —R⁴, —R⁵ are each —H, the following provisos apply:

(i) —R¹, —R², and —R³ are not all —OMe;

(ii) —R¹, —R², and —R³ are not all —F;

(ii) —R² is not —OCF₃, where each of —R¹ and —R³ is —H; and

(iv) —R² is not —H, where each of —R¹ and —R³ is —F.

In some embodiments, —R¹, —R² and —R³ are the same.

In some embodiments —R¹ and —R³ are the same.

In some embodiments, each of —R², —R³ and —R⁴ is independently selected from —H, —F, —Cl, —Br, —I, and —OR^{A1}.

In some embodiments, each -Q¹ is independently selected from: —F, —Cl, —R^{A}, and —OR^{A}.

In some embodiments, each of —R¹, —R² and —R³ is —OR^{A}.

In some embodiments, each —R^{A} is independently methyl, which may be substituted by one or more fluoro or chloro groups.

In some embodiments, each —R^{A} is unsubstituted methyl.

In some embodiments, one of —R⁴ and —R⁵ is —H.

In some embodiments, both of —R⁴ and —R⁵ is —H.

In some embodiments, one of —R⁴ and —R⁵ is —R^{B}.

Particularly suitable are prodrugs of compound (XI) wherein one or more of the hydroxy groups is esterified to an —O—C(═O)—R^{C} group, where R^{C} is selected from methyl and ethyl.

In some embodiments, R^{C} is methyl.

In some embodiments, R² is a hydroxy group and is the only hydroxy esterified to an —O—C(═O)-Me group. In other embodiments, R² is a hydroxy group and all the hydroxy groups are esterified to an —O—C(═O)-Me group.

Of particular relevance are the prodrug compounds:

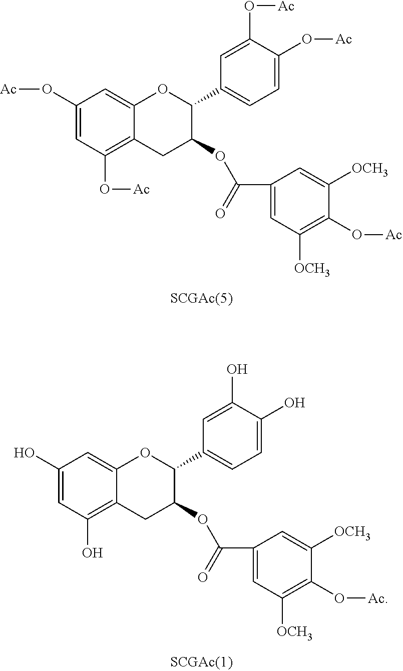 [SMILES: COc1cc(C(=O)O[C@H]2Cc3c(OC(C)=O)cc(OC(C)=O)cc3O[C@@H]2c2ccc(OC(C)=O)c(OC(C)=O)c2)cc(OC)c1OC(C)=O ; COc1cc(C(=O)O[C@H]2Cc3c(O)cc(O)cc3O[C@@H]2c2ccc(O)c(O)c2)cc(OC)c1OC(C)=O]

Also of interest are the following compounds of formula (X):

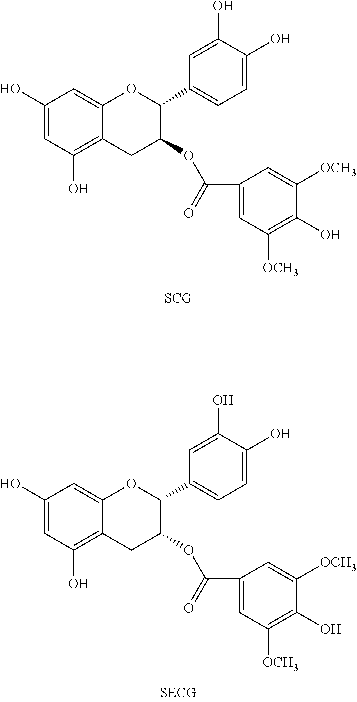 [SMILES: COc1cc(C(=O)O[C@H]2Cc3c(O)cc(O)cc3O[C@@H]2c2ccc(O)c(O)c2)cc(OC)c1O ; COc1cc(C(=O)O[C@@H]2Cc3c(O)cc(O)cc3O[C@@H]2c2ccc(O)c(O)c2)cc(OC)c1O]

In some embodiments, the compounds are of formula (XIa):

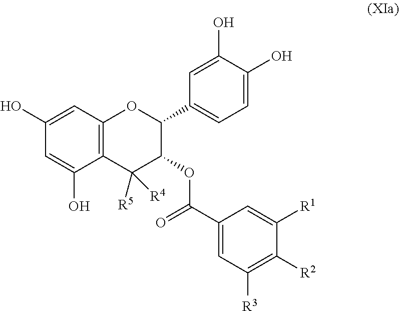

In other embodiments, the compounds are of formula (XIb):

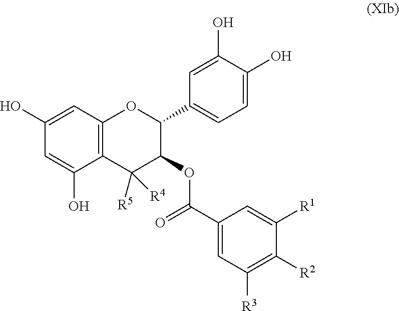

Preferably, the compound is a compound of formula (XI) or an isomer thereof.

Accordingly, the compounds of formula (X) have the structure below:

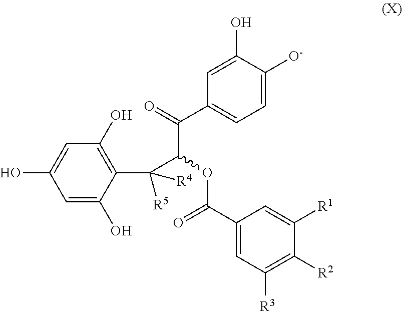

—R¹, —R², —R³, —R⁴ and —R⁵ are defined according to the compound of formula (XI) or an isomer, salt, solvate or prodrug thereof.

A reference to a compound of formula (X) also includes reference to the canonical forms of the structure shown. For example, reference to the compound of formula (II) includes reference to the compound of formula (III):

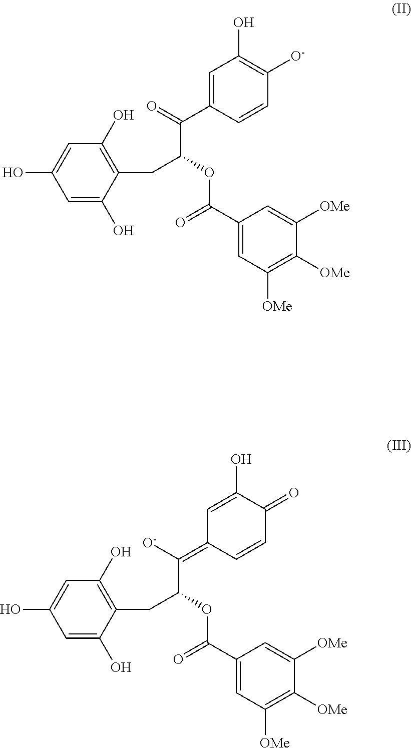 [SMILES: COc1cc(C(=O)O[C@H](Cc2c(O)cc(O)cc2O)C(=O)c2ccc([O-])c(O)c2)cc(OC)c1OC ; COc1cc(C(=O)O[C@H](Cc2c(O)cc(O)cc2O)/C([O-])=C2\C=CC(=O)C(O)=C2)cc(OC)c1OC]

Suitable methods for the synthesis of the compounds of formula (X) and (XI) are known in the prior art. See for example, WO 2009/081275.

In a general aspect the invention provides a method of treatment, including a method of treating cancer, such as melanoma, which may comprise administering a therapeutically effective dose of a Tyrosinase-activated prodrug and a tyrosinase expression enhancer, such as MTX, to an individual in need thereof. The tyrosinase-activated prodrug may be for the treatment of cancer, such as melanoma.

Another aspect of the invention provides a method of treating melanoma or other cancer, which may comprise administering a therapeutically effective dose of a catechin compound, such as a compound of formula (XI), and a tyrosinase expression enhancer, such as MTX, to an individual in need thereof. Alternative tyrosinase expression enhancers, such as U0126, PD0325901, and PLX4720 may be used in place of MTX.

Related aspects provide a catechin compound, such as a compound of formula (XI), and a tyrosinase expression enhancer, such as MTX, for use in the treatment of melanoma or other cancer, and the use of a catechin compound, such as a compound of formula (XI), and a tyrosinase expression enhancer, such as MTX, in the manufacture of a medicament for use in the treatment of melanoma or other cancer.

Also provided is a method of preventing or inhibiting the dissemination of a cancer in a subject from one organ to another organ. The present inventors have established that the combination of the present invention inhibits invasiveness. Blocking invasion may be achieved through MTX-mediated MITF stimulated differentiation of invasive stem-like cells. Once differentiated, stem-like cells lose their invasive properties.

In particular, the combination of a catechin compound and a tyrosinase expression enhancer may be used to limit or prevent dissemination of a cancer, particularly melanoma, to the liver, such as from the spleen. The liver is one of the preferential metastatic locations for melanoma, and the combination of the invention may be used advantageously to limit the spread of melanoma to this organ. Additionally, the combination of a catechin compound and a tyrosinase expression enhancer may be used in a method of treatment including the step of a blocking brain metastasis.

Notably, the combination treatments described herein are also suitable for use against those melanomas with BRAF, NRAS, p53, GNAQ, EGFR, PDGFR, RAC or c-kit mutations, or any combination of mutants thereof.

In further aspects of the present invention there is provided a method of treatment which leads to the differentiation of a stem-like tumor cell, such as melanoma stem-like cell. The method may comprise administering to a subject a compound which differentiates a stem-like tumor cell into a matured cell that is a tyrosinase producer. The method also includes the step of administering to the subject a catechin compound, as described herein.

The compound for use in the differentiation may be MTX.

Tumors comprise multiple phenotypically distinct subpopulations of cells, some of which are proposed to possess stem cell-like properties, being able to self-renew, seed and maintain tumors, and provide a reservoir of therapeutically-resistant cells. Although at any given moment cells within a tumor may exhibit differentiated, proliferative or invasive phenotypes, an ability to switch phenotypes implies that most cells will have the potential to adopt an invasive stem cell-like identity.

For melanoma, one of the most obvious characteristics of stem-like cells in tumors (“stem-like” since they will bear activating mutations in oncogenes such as BRAF not found in physiological stem cells) is reduced MITF expression. Typically normal physiological melanocyte stem cells have low MITF activity, and high MITF activity is characteristic of differentiated cells. Consistent with this, stem-like cells can be found at a low frequency (0.1 to 5% of the population) in cultured melanoma cells. Such cells are highly tumorigenic, express low levels of MITF and are slow dividing. Such cells are referred to in the art as label-retaining cells (see Cheli 2011 and 2011).

The worked examples provided herein describe methods for determining the level of tyrosinase production in a cell. Thus, it is possible to determine whether a cell has been converted to a tyrosinase producer.

The worked examples provided herein describe methods for determining MITF levels in a cell. MITF-low melanoma cells with stem-like properties may also be identified by immunofluorescence in melanoma tissue samples, such as human melanoma tissue samples. Thus, it is possible to determine whether MITF levels in a cell have increased in response to a compound which differentiates a stem-like tumor cell into a matured cell that is a tyrosinase producer.

Thus, in one embodiment, tumor cell differentiation, such as melanoma cell differentiation, is associated with an increase in MITF activity and/or the appearance of, or the increase in, tyrosinase activity.

The present invention provides methods for the treatment of cancer. In preferred embodiments, the cancer is melanoma. The melanoma may be a metastatic melanoma or a melanoma with somatic mutation or phenotypic resistance.

Melanoma is a malignant neoplasm of melanocytes in the skin. Melanoma which may be treated as described herein may include, for example, superficial spreading melanoma, nodular melanoma, acral lentiginous melanoma or lentigo maligna (melanoma); and metastatic melanoma, for example melanoma displaying local or distant metastases. The melanoma may be at any stage. For example, the melanoma may be stage 0, I, II, III or IV melanoma as described by Balch (see Balch et al.). Stage IV, metastatic melanoma, is a melanoma that has spread to other sites of the body. The spread occurs through the lymphatic system and/or the blood vessels. In some instances stem-like tumor cells contribute to metastatic melanoma.

In addition to melanoma, the compounds and combinations described may also be useful in the treatment of other forms of cancer, for example breast cancer. Expression of MITF in breast cancer cells is likely to induce expression of tyrosinase. For example, MITF may be activated in some breast cancers using a demethylating agent. In such cases, the methods of treatment described herein would be suitable for use in the treatment of breast cancer.

In some embodiments, the cancer may be a metastatic cancer.

Melanocytes are derived from pluripotent neural crest stem cells. Melanocyte development is modulated by KIT and MITF, factors that are mutated and/or amplified oncogenes in many cases of melanoma (see Chin et al.). Mutations in c-kit are seen in approximately 15 to 20% of patients with advanced melanomas.

The MAPK pathway is activated in almost all melanomas (see Omholt et al.). In non-malignant cells the interaction between a growth factor receptor and its ligand is required to activate this pathway. This leads to a series of events that promote cellular growth and survival. The RAS family members are G-proteins, which serve as critical mediators in the transduction of such signals. BRAF mutations are most frequently detected in cutaneous melanoma (in 40-50% of cases). NRAS mutations have been identified in 10 to 15% of cutaneous melanomas and are thought to be an important driver of oncogenesis. A somatic mutation in the NRAS gene can cause constitutive activity of the NRAS protein that leads to the serial activation of serine/threonine kinases. The consequence of constitutive activation of NRAS-mediated pathway is cell proliferation, cellular transformation and enhanced cell survival. The conversion of a normal cell into a highly proliferative transformed cell may be mediated through the overexpression and/or hyperactivation of various growth factor receptors, such as c-Met, epidermal growth factor receptor (EGFR), and KIT (see Bardeesy et al.).

The prognosis of patients with advanced melanoma is influenced by the specific mutations present in a specific tumor. Melanomas with somatic mutations of either NRAS or BRAF are associated with a poorer prognosis. Patients with acral or mucosal melanoma that contain KIT mutations have a poorer prognosis compared with similar patients whose tumors do not contain identifiable KIT mutations. In patients with ocular melanoma, somatic mutations in either GNAQ or GNA11 do not appear to be associated with poor prognosis (at least relative to the small group of patients with tumors lacking either mutation). Mutations within the BAP1 gene, which is thought to regulate cellular growth control, does appear to be associated with increased risk of metastasis and worsened prognosis.

In one embodiment, a cancer, such as melanoma, is one associated with a somatic mutation.

In one embodiment, the somatic mutation is associated with the activation of the MAP kinase pathway or activation of a bypass requirement for the MAP kinase pathway.

The present invention provides methods for the treatment of melanoma, including those melanomas that contain mutations in one or more of BRAF, NRAS, p53, GNAQ, EGFR, PDGFR, RAC and c-kit. The combination of a tyrosinase expression enhancer and a catechin compound may act through a mechanism that is independent of these mutant proteins. The combination therefore provides an alternative to those treatment strategies that target signalling pathways featuring these mutants.

It has been reported that BRAF somatic missense mutations occur in 66% of malignant melanomas, as well as at lower frequencies in other cancers (see Davies et al.). The BRAF mutations are generally in the kinase domain, and a single substitution of glutamic acid for valine at amino acid 600 (V600E mutation) accounts for 80% of these mutations with most of the remainder consisting of an alternate substitution at the V600 locus (V to K). V600E substitution causes increased kinase activity. Notably, the V600E substitution is associated with malignant melanoma Inhibitors of BRAF V600E, such as Plexxikon 4032 (PLX-4032, also known as RG7204 and vermurafenib provide limited therapeutic benefits.

The majority of patients treated with an inhibitor of BRAF eventually succumb to disease progression (see Chapman et al., 2011). Resistance the BRAF inhibitor is most results from a bypass mechanism within the MAPK pathway that can restore ERK activation (Nazarian et al.), synthesis of truncated protein (Poulikakos et al.), or signalling through the parallel growth and survival PI3K pathway (Villanueva et al., 2010). Furthermore, only partial positive responses are observed in patients treated with c-kit inhibitors.

Thus, in one embodiment, the cancer is one in which BRAF carries one or more mutations.

The invention provides the use of a combination of a tyrosinase expression enhancer and a catechin compound to treat melanoma. The method of treatment may be independent of the BRAF biochemical pathway, including those proteins acting downstream of the BRAF pathway, such as MEK1/MEK2. The present invention also provides use of a combination of a tyrosinase expression enhancer and a catechin compound for the treatment of melanomas containing genetic risk factors such as those described above. The combination of a tyrosinase expression enhancer and a catechin compound may be a combination of MTX and TMECG.

Thus, in one embodiment, the cancer is one in which MEK1 or MEK2 carries a mutation.

The methods of treating cancer, such as melanoma, described herein may be used where a BRAF mutant is present. In one embodiment the subject for treatment may be a subject that has been treated with a BRAF-inhibiting drug. In one embodiment the subject is one for whom the treatment with such a drug is no longer effective.

In one embodiment, the BRAF mutant is one where the mutation is found at one or more of positions 461, 462, 463, 465, 468, 580, 585, 593, 594, 595, 596, 598, 599, 600 and 727. The mutation may be an activating mutation. A number of mutations in BRAF are known. In particular, the V600E mutation is prominent. Other mutations which have been found are R461I, I462S, G463E, G463V, G465A, G465E, G465V, G468A, G468E, N580S, E585K, D593V, F594L, G595R, L596V, T598I, V599D, V599E, V599K, V599R, K600E, A727V, and most of these mutations are clustered to two regions: the glycine-rich P loop of the N lobe and the activation segment and flanking regions. In one embodiment, BRAF carries one or more the specific mutations listed above. In one embodiment, the BRAF mutant is V600E.

A reference to a BRAF mutant may include a reference to those BRAF proteins lacking one or more of exons 4, 5, 6, 7 and 8, such as all of exons 4-8, such as p61BRAF(V600E) which is the 61 kDa form of BRAF(V600E). The region associated with exons 4-8 encompasses the RAS-binding domain (see Poulikakos et al.).

BRAF inhibiting drugs are well known in the art and are designated as such by their supplier. BRAF inhibitors include PDC-4032, GSK218436 and PLX-3603 (also known as RO5212054). Additionally, inhibitors that block MEK1/MEK2 kinase downstream of the BRAF pathway include Trametinib, Selumetinib and MEK162.

The methods of treating cancer, such as melanoma, described herein may be used where a c-kit mutant is present. In one embodiment the subject for treatment may be a subject that has been treated with a c-kit-inhibiting drug. In one embodiment the subject is one for whom the treatment with such a drug is no longer effective.

Mutations in c-kit are seen in approximately 15 to 20% of patients with advanced melanomas. Kit inhibitors, imatinib and nilotinib, target patients with activating mutations in the c-kit gene. Methods to identify c-kit mutations and other genetic risk factors including p53 mutations, NRAS mutations are well known in the art.

The present invention also provides methods of treating a cell, such as cancer cell, with a tyrosinase expression enhancer and/or a catechin compound. The cancer cell may be treated or contacted in vitro or in vivo. In some preferred embodiments, the cancer cell is a melanoma cell.

In one embodiment, the cancer cell is a melanoma cell having a kinase-activating mutation, such as a mutation in one or more of BRAF, NRAS, p53, GNAQ, EGFR, PDGFR, RAC and c-kit.

The cancer cell may be a melanoma cell having a BRAF mutation, such as BRAF V600E, or one or more of the other BRAF mutations mentioned above. The cell may be resistant to one or more BRAF inhibitors.

In one embodiment, the cancer cell is a melanoma cell that has developed phenotypic resistance to chemotherapy.

Combinations of a tyrosinase expression enhancer, such as MTX, and a catechin compound, such as TMECG, as described herein, may be the sole therapeutic agents which are administered to the individual or they may be administered in combination with one or more additional active compounds.

Methods of measuring the effect of a combination of compounds as described above on melanoma or other cancer cells are well-known in the art and are exemplified herein. For example, the effect of combinations of tyrosinase expression enhancer and a catechin compound, such as TMECG, on the cell death in melanoma or other cancer cells may be determined by contacting a population of melanoma or other cancer cells with the combination, preferably in the form of a pharmaceutically acceptable composition(s), and determining the amount of cell death in the population. An increase in cell death in the cancer cell population treated with the combination, relative to untreated cancer cells or cancer cells treated with either one of the compounds individually, is indicative that the combination has a cytotoxic effect on the cancer cells. Suitable methods may be practiced in vitro or in vivo.

The term “treatment” as used herein in the context of treating a cancer condition, such as melanoma, pertains generally to treatment and therapy, whether of a human or an animal (e.g. in veterinary applications), in which some desired therapeutic effect is achieved, for example, the inhibition or delay of the progress of the condition, and includes a reduction in the rate of progress, a halt in the rate of progress, amelioration of the condition, and cure of the condition. Treatment as a prophylactic measure (i.e. prophylaxis) is also included. For example, an individual susceptible to or at risk of the occurrence or re-occurrence of melanoma may be treated as described herein. Such treatment may prevent or delay the occurrence or re-occurrence of melanoma in the individual.

The compounds described herein may be administered in therapeutically-effective amounts.

The term “therapeutically-effective amount” as used herein, pertains to that amount of an active compound, or a combination, material, composition or dosage form which may comprise an active compound, which is effective for producing some desired therapeutic effect, commensurate with a reasonable benefit/risk ratio.

While it is possible for the compounds in a combination described above to be administered alone, it is preferable to present the compounds in the same or separate pharmaceutical compositions (e.g. formulations) which may comprise the compound(s) as defined above, together with one or more pharmaceutically acceptable carriers, adjuvants, excipients, diluents, fillers, buffers, stabilisers, preservatives, lubricants, or other materials well known to those skilled in the art. Optionally, other therapeutic or prophylactic agents may be included.

For example, methionine cycle inhibitors; adenosine metabolism inhibitors; equilibrate nucleoside transporters inhibitors and/or adenosine deaminase inhibitors as described above may be included in the pharmaceutical compositions.

The present invention further provides pharmaceutical compositions, as defined above, and methods of making a pharmaceutical composition which may comprise admixing a tyrosinase expression enhancer and a catechin compound, such as TMECG, together with one or more pharmaceutically acceptable carriers, excipients, buffers, adjuvants, stabilisers, or other materials, as described herein.

The tyrosinase expression enhancer and the catechin compound may be formulated in separate pharmaceutical compositions, which compositions are suitable for administering the tyrosinase expression enhancer and the catechin compound separately or simultaneously.

The term “pharmaceutically acceptable” as used herein pertains to compounds, materials, compositions, and/or dosage forms which are, within the scope of sound medical judgement, suitable for use in contact with the tissues of a subject (e.g. human) without excessive toxicity, irritation, allergic response, or other problem or complication, commensurate with a reasonable benefit/risk ratio. Each carrier, excipient, etc. must also be “acceptable” in the sense of being compatible with the other ingredients of the formulation.

Suitable carriers, excipients, etc. can be found in standard pharmaceutical texts, for example, Remington's Pharmaceutical Sciences, 18th edition, Mack Publishing Company, Easton, Pa., 1990.

The formulations may conveniently be presented in unit dosage form and may be prepared by any methods well known in the art of pharmacy. Such methods include the step of bringing into association the active compound with the carrier which constitutes one or more accessory ingredients. In general, the formulations are prepared by uniformly and intimately bringing into association the active compound with liquid carriers or finely divided solid carriers or both, and then if necessary shaping the product.

Formulations may be in the form of liquids, solutions, suspensions, emulsions, elixirs, syrups, tablets, losenges, granules, powders, capsules, cachets, pills, ampoules, suppositories, pessaries, ointments, gels, pastes, creams, sprays, mists, foams, lotions, oils, boluses, electuaries, or aerosols.

The active compound or pharmaceutical composition which may comprise the active compound may be administered to a subject by any convenient route of administration, whether systemically/peripherally or at the site of desired action, including but not limited to, oral (e.g. by ingestion); topical (including e.g. transdermal, intranasal, ocular, buccal, and sublingual); pulmonary (e.g. by inhalation or insufflation therapy using, e.g. an aerosol, e.g. through mouth or nose); rectal; vaginal; parenteral, for example, by injection, including subcutaneous, intradermal, intramuscular, intravenous, intraarterial, intracardiac, intrathecal, intraspinal, intracapsular, subcapsular, intraorbital, intraperitoneal, intratracheal, subcuticular, intraarticular, subarachnoid, and intrasternal; by implant of a depot, for example, subcutaneously or intramuscularly.

Formulations suitable for oral administration (e.g. by ingestion) may be presented as discrete units such as capsules, cachets or tablets, each containing a predetermined amount of the active compound; as a powder or granules; as a solution or suspension in an aqueous or non-aqueous liquid; or as an oil-in-water liquid emulsion or a water-in-oil liquid emulsion; as a bolus; as an electuary; or as a paste.

A tablet may be made by conventional means, e.g., compression or moulding, optionally with one or more accessory ingredients. Compressed tablets may be prepared by compressing in a suitable machine the active compound in a free-flowing form such as a powder or granules, optionally mixed with one or more binders (e.g. povidone, gelatin, acacia, sorbitol, tragacanth, hydroxypropylmethyl cellulose); fillers or diluents (e.g. lactose, microcrystalline cellulose, calcium hydrogen phosphate); lubricants (e.g. magnesium stearate, talc, silica); disintegrants (e.g. sodium starch glycolate, cross-linked povidone, cross-linked sodium carboxymethyl cellulose); surface-active or dispersing or wetting agents (e.g. sodium lauryl sulfate); and preservatives (e.g. methyl p-hydroxybenzoate, propyl p-hydroxybenzoate, sorbic acid). Moulded tablets may be made by moulding in a suitable machine a mixture of the powdered compound moistened with an inert liquid diluent. The tablets may optionally be coated or scored and may be formulated so as to provide slow or controlled release of the active compound therein using, for example, hydroxypropylmethyl cellulose in varying proportions to provide the desired release profile. Tablets may optionally be provided with an enteric coating, to provide release in parts of the gut other than the stomach.

Formulations suitable for topical administration (e.g. transdermal, intranasal, ocular, buccal, and sublingual) may be formulated as an ointment, cream, suspension, lotion, powder, solution, past, gel, spray, aerosol, or oil. Alternatively, a formulation may comprise a patch or a dressing such as a bandage or adhesive plaster impregnated with active compounds and optionally one or more excipients or diluents.

Formulations suitable for topical administration in the mouth include losenges which may comprise the active compound in a flavoured basis, usually sucrose and acacia or tragacanth; pastilles which may comprise the active compound in an inert basis such as gelatin and glycerin, or sucrose and acacia; and mouthwashes which may comprise the active compound in a suitable liquid carrier.

Formulations suitable for topical administration to the eye also include eye drops wherein the active compound is dissolved or suspended in a suitable carrier, especially an aqueous solvent for the active compound.

Formulations suitable for nasal administration, wherein the carrier is a solid, include a coarse powder having a particle size, for example, in the range of about 20 to about 500 microns which is administered in the manner in which snuff is taken, i.e. by rapid inhalation through the nasal passage from a container of the powder held close up to the nose. Suitable formulations wherein the carrier is a liquid for administration as, for example, nasal spray, nasal drops, or by aerosol administration by nebuliser, include aqueous or oily solutions of the active compound.

Formulations suitable for administration by inhalation include those presented as an aerosol spray from a pressurised pack, with the use of a suitable propellant, such as dichlorodifluoromethane, trichlorofluoromethane, dichoro-tetrafluoroethane, carbon dioxide, or other suitable gases.

Formulations suitable for topical administration via the skin include ointments, creams, and emulsions. When formulated in an ointment, the active compound may optionally be employed with either a paraffinic or a water-miscible ointment base. Alternatively, the active compounds may be formulated in a cream with an oil-in-water cream base. If desired, the aqueous phase of the cream base may include, for example, at least about 30% w/w of a polyhydric alcohol, i.e., an alcohol having two or more hydroxyl groups such as propylene glycol, butane-1, 3-diol, mannitol, sorbitol, glycerol and polyethylene glycol and mixtures thereof. The topical formulations may desirably include a compound which enhances absorption or penetration of the active compound through the skin or other affected areas. Examples of such dermal penetration enhancers include dimethylsulfoxide and related analogues.

When formulated as a topical emulsion, the oily phase may optionally comprise merely an emulsifier (otherwise known as an emulgent), or it may comprise a mixture of at least one emulsifier with a fat or an oil or with both a fat and an oil. Preferably, a hydrophilic emulsifier is included together with a lipophilic emulsifier which acts as a stabiliser. It is also preferred to include both an oil and a fat. Together, the emulsifier(s) with or without stabiliser(s) make up the so-called emulsifying wax, and the wax together with the oil and/or fat make up the so-called emulsifying ointment base which forms the oily dispersed phase of the cream formulations.

Suitable emulgents and emulsion stabilisers include Tween 60, Span 80, cetostearyl alcohol, myristyl alcohol, glyceryl monostearate and sodium lauryl sulphate. The choice of suitable oils or fats for the formulation is based on achieving the desired cosmetic properties; since the solubility of the active compound in most oils likely to be used in pharmaceutical emulsion formulations may be very low. Thus the cream should preferably be a non-greasy, non-staining and washable product with suitable consistency to avoid leakage from tubes or other containers. Straight or branched chain, mono- or dibasic alkyl esters such as di-isoadipate, isocetyl stearate, propylene glycol diester of coconut fatty acids, isopropyl myristate, decyl oleate, isopropyl palmitate, butyl stearate, 2-ethylhexyl palmitate or a blend of branched chain esters known as Crodamol CAP may be used, the last three being preferred esters. These may be used alone or in combination depending on the properties required.

Alternatively, high melting point lipids such as white soft paraffin and/or liquid paraffin or other mineral oils can be used.

Formulations suitable for rectal administration may be presented as a suppository with a suitable base which may comprise, for example, cocoa butter or a salicylate.

Formulations suitable for vaginal administration may be presented as pessaries, tampons, creams, gels, pastes, foams or spray formulations containing in addition to the active compound, such carriers as are known in the art to be appropriate.

Formulations suitable for parenteral administration (e.g. by injection, including cutaneous, subcutaneous, intramuscular, intravenous and intradermal), include aqueous and non-aqueous isotonic, pyrogen-free, sterile injection solutions which may contain anti-oxidants, buffers, preservatives, stabilisers, bacteriostats, and solutes which render the formulation isotonic with the blood of the intended recipient; and aqueous and non-aqueous sterile suspensions which may include suspending agents and thickening agents, and liposomes or other microparticulate systems which are designed to target the compound to blood components or one or more organs. Examples of suitable isotonic vehicles for use in such formulations include Sodium Chloride Injection, Ringer's Solution, or Lactated Ringer's Injection. Typically, the concentration of the active compound in the solution is from about 1 ng/mL to about 10 μg/mL, for example from about 10 ng/mL to about 1 μg/mL. The formulations may be presented in unit-dose or multi-dose sealed containers, for example, ampoules and vials, and may be stored in a freeze-dried (lyophilised) condition requiring only the addition of the sterile liquid carrier, for example water for injections, immediately prior to use. Extemporaneous injection solutions and suspensions may be prepared from sterile powders, granules, and tablets. Formulations may be in the form of liposomes or other microparticulate systems which are designed to target the active compound to blood components or one or more organs.

It will be appreciated that appropriate dosages of the active compounds, and compositions which may comprise the active compounds, can vary from patient to patient. Determining the optimal dosage will generally involve the balancing of the level of therapeutic benefit against any risk or deleterious side effects of the treatments of the present invention. The selected dosage level will depend on a variety of factors including, but not limited to, the activity of the particular compound, the route of administration, the time of administration, the rate of excretion of the compound, the duration of the treatment, other drugs, compounds, and/or materials used in combination, and the age, sex, weight, condition, general health, and prior medical history of the patient. The amount of compound and route of administration will ultimately be at the discretion of the physician, although generally the dosage will be to achieve local concentrations at the site of action which achieve the desired effect without causing substantial harmful or deleterious side-effects.

In general, a suitable dose of each active compound is in the range of about 100 μg to about 250 mg per kilogram body weight of the subject per day. Where the active compound is a salt, an ester, prodrug, or the like, the amount administered is calculated on the basis of the parent compound and so the actual weight to be used is increased proportionately.

For example, in some embodiments, 0.1 to 10 mg/kg/day, preferably 1 mg/kg/day of the tyrosinase expression enhancer, such as MTX, and 1 to 100 mg/kg/day, preferably 50 mg/kg/day of a catechin compound, such as TMECG/TMCG, may be used to reduce melanoma tumors.

Administration in vivo can be effected in one dose, continuously or intermittently (e.g. in divided doses at appropriate intervals) throughout the course of treatment. Methods of determining the most effective means and dosage of administration are well known to those of skill in the art and will vary with the formulation used for therapy, the purpose of the therapy, the target cell being treated, and the subject being treated. Single or multiple administrations can be carried out with the dose level and pattern being selected by the treating physician.

The administration of the tyrosinase expression enhancer and the catechin compound may be in one combined dose, continuously or intermittently. Single or multiple administrations may be carried out. Alternatively, the tyrosinase expression enhancer and the catechin compound may be administered separately, where each is independently administered in one dose, continuously or intermittently.

Other aspects of the invention relate to methods of screening for compounds which modulate, such as increase, tyrosinase expression in a cancer cell, such as a melanoma cell.

A method may comprise: contacting a cancer cell with and a catechin compound in the presence and absence of a test compound and determining the quantity of the QM form of the catechin compound. The cancer cell may be a melanoma cell. Additionally or alternatively, the method may comprise the step of determining the quantity of tyrosinase mRNA or protein, as described herein.

Further aspects of the invention relate to methods of screening for compounds which modulate, such as increase, MITF expression in a cancer cell, such as a melanoma cell.

A method may comprise: contacting a cancer cell with a catechin compound in the presence and absence of a test compound and determining the quantity of the QM form of the catechin compound. The cancer cell may be a melanoma cell. Additionally or alternatively, the method may comprise the step of determining the quantity of MITF mRNA or protein, as described herein.

Optionally, each of the screening methods above may be undertaken in conjunction with each other, and/or in conjunction with a method of screening for compounds which inhibit DHFR. This screening method may be conducted prior to the methods described above. Thus, compounds having DHFR inhibitory activity may be identified and then tested in a further screening method to determine that compound's ability to modulate, such as increase, tyrosinase expression or MITF expression.

The methods may be conducted in vitro or in vivo.

A cancer cell may be a cell where tyrosinase is expressed.

The melanoma cell may be a SK-Mel-28 cell.

Other aspects of the invention relate to methods of screening for compounds which differentiate stem-like cells, such as stem-like melanoma cells. The differentiation of the stem-like cell may be determined by an increase in MITF expression and/or the increase in Tyrosinase expression.

The method may comprise: contacting a stem-like cell with a test compound and determining the quantity of MITF mRNA or protein and/or the quantity of TYR mRNA or protein, as described herein. The quantity of MITF mRNA or protein and/or the quantity of TYR mRNA or protein may be compared with the quantities produced in the absence of the test compound. In one embodiment, the method may additionally comprise contacting the stem-like cell with a catechin compound. The quantity of TYR mRNA or protein may be inferred from a change in the conversion of the catechin compound to its QM form compared to the conversion in the absence of the test compound.

The methods of treatment may comprise the step of administering active agents to an individual in need of treatment.

An individual suitable for treatment as described above may be a mammal, such as a rodent (e.g. a guinea pig, a hamster, a rat, a mouse), murine (e.g. a mouse), canine (e.g. a dog), feline (e.g. a cat), equine (e.g. a horse), a primate, simian (e.g. a monkey or ape), a monkey (e.g. marmoset, baboon), an ape (e.g. gorilla, chimpanzee, orangutan, gibbon), or a human. In some preferred embodiments, the individual is a human.

In some embodiments, the individual is a rodent.

Alternatively, the individual may be non-human.

The individual may be a subject having cancer or at risk thereof.

Where a subject has cancer, the cancer is one where tyrosinase is expressed or expressible within a cancer cell. The cancer may be melanoma.

In one embodiment, the subject may have melanoma in which one or more of BRAF, NRAS, p53, GNAQ, EGFR, PDGFR, RAC or c-kit carries a mutation. The subject may have melanoma in which BRAF carries a mutation, such as any one of the mutations described in the Cancer section above,

The patient may be a patient having melanoma and has previously undergone treatment, for example with an alternative melanoma treatment regime.

The patient may be a cancer patient, such as a melanoma patient, who has developed resistance to a cancer drug. For example, the patient may have been previously treated with BRAF inhibiting drugs. In one embodiment, the patient may be one for whom the treatment with such drugs is not or is no longer effective.

In one embodiment, the patient may have been previously treated with a MEK inhibiting drug, such as a MEK1 or MEK2 inhibiting drug.

For example, the patient may have previously been treated with vemurafenib, which targets BRAF, with resistance to that treatment arising from mutations that bypass the requirement for BRAF in the MAP kinase signalling pathway. The present invention therefore provides an alternative strategy for treating drug-resistant cancers.

The present inventors have established that the methods of treatment described herein may also be performed on subjects regardless of the BRAF, NRAS, p53, GNAQ, EGFR, PDGFR, RAC and/or c-kit status. Thus, the methods of treatment may be for those subjects having cancer, such as melanoma, where the BRAF, NRAS, p53, GNAQ, EGFR, PDGFR, RAC and/or c-kit show a wild type status. The present invention therefore provides an alternative to those treatments that are based on the administration of active agents that target mutant forms of BRAF, PTEN, NRAS and/or p53.

Many anticancer therapeutic treatments use compounds that target proteins carrying a mutation, such as BRAF mutant and p53 mutants. However, not all cancers are linked to proteins carrying such mutations, and therefore the use of the targeted anticancer compounds in these situations may not be useful. The present invention may be used to treat those patients whose cancers where one or more of BRAF, NRAS, p53, GNAQ, EGFR, PDGFR, RAC or c-kit do not carry a mutation.

Unless otherwise specified, references to a compound herein also include isomeric, ionic, salt, solvate, and protected forms of the compound. For example, a reference to a hydroxyl group also includes the anionic form (—O—), a salt or solvate thereof, as well as conventional protected forms of a hydroxyl group. Ionic forms, salts, solvates, and protected forms of any particular compound are readily apparent to the skilled person.

Certain compounds may exist in one or more particular geometric, optical, enantiomeric, diasteriomeric, epimeric, stereoisomeric, tautomeric, conformational, or anomeric forms, including but not limited to, cis- and trans-forms; E- and Z-forms; c-, t-, and r-forms; endo- and exo-forms; R-, S-, and meso-forms; D- and L-forms; d- and l-forms; (+) and (−) forms; keto, enol-, and enolate-forms; syn- and anti-forms; synclinal- and anticlinal-forms; α- and β-forms; axial and equatorial forms; boat-, chair-, twist-, envelope-, and half chair-forms; and combinations thereof, hereinafter collectively referred to as “isomers” (or “isomeric forms”).

Note that, except as discussed below for tautomeric forms, specifically excluded from the term “isomers”, as used herein, are structural (or constitutional) isomers (i.e. isomers which differ in the connections between atoms rather than merely by the position of atoms in space). For example, a reference to a methoxy group, OCH₃, is not to be construed as a reference to its structural isomer, a hydroxymethyl group, CH₂OH. Similarly, a reference to ortho-chlorophenyl is not to be construed as a reference to its structural isomer, meta-chlorophenyl. However, a reference to a class of structures may well include structurally isomeric forms falling within that class (e.g., C₁₋₇ alkyl includes n propyl and iso-propyl; butyl includes n-, iso-, sec-, and tert-butyl; methoxyphenyl includes ortho-, meta-, and para-methoxyphenyl).

The above exclusion does not pertain to tautomeric forms, for example, keto-, enol-, and enolate-forms, as in, for example, the following tautomeric pairs: keto/enol (illustrated below), imine/enamine, amide/imino alcohol, amidine/amidine, nitroso/oxime, thioketone/enethiol, N-nitroso/hyroxyazo, and nitro/aci-nitro.

Note that specifically included in the term “isomer” are compounds with one or more isotopic substitutions. For example, H may be in any isotopic form, including ¹H, ²H (D), and ³H (T); C may be in any isotopic form, including ¹²C, ¹³C, and ¹⁴C; O may be in any isotopic form, including ¹⁶O and ¹⁸O; and the like.

Unless otherwise specified, a reference to a particular compound includes all such isomeric forms, including (wholly or partially) racemic and other mixtures thereof. Methods for the preparation (e.g. asymmetric synthesis) and separation (e.g., fractional crystallisation and chromatographic means) of such isomeric forms are either known in the art or are readily obtained by adapting the methods taught herein, or known methods, in a known manner.

It may be convenient or desirable to prepare, purify, and/or handle a corresponding salt of the active compound, for example, a pharmaceutically-acceptable salt. Examples of pharmaceutically acceptable salts are discussed in Berge et al., 1977, “Pharmaceutically Acceptable Salts”, J. Pharm. Sci., Vol. 66, pp. 1-19.

The compounds of formula (X) may be ionic, typically anionic. Where the compound is ionic, there may be a pharmaceutically acceptable counter ion. Where such a counter ion is present, the compounds of formula (X) may be referred to as pharmaceutically acceptable salts.

The compounds of the invention may also be zwitterionic.

For example, if the compound is anionic, or has a functional group which may be anionic (e.g., COOH may be COO⁻), then a salt may be formed with a suitable cation. Examples of suitable inorganic cations include, but are not limited to, alkali metal ions such as Na⁺ and K+, alkaline earth cations such as Ca²⁺ and Mg²⁺, and other cations such as Al³⁺. Examples of suitable organic cations include, but are not limited to, ammonium ion (i.e., NH₄⁺) and substituted ammonium ions (e.g., NH₃R⁺, NH₂R₂⁺, NHR₃⁺, NR₄⁺). Examples of some suitable substituted ammonium ions are those derived from: ethylamine, diethylamine, dicyclohexylamine, triethylamine, butylamine, ethylenediamine, ethanolamine, diethanolamine, piperazine, benzylamine, phenylbenzylamine, choline, meglumine, and tromethamine, as well as amino acids, such as lysine and arginine. An example of a common quaternary ammonium ion is N(CH₃)⁴⁺.

If the compound is cationic, or has a functional group which may be cationic (e.g., NH₂ may be NH₃⁺), then a salt may be formed with a suitable anion. Examples of suitable inorganic anions include, but are not limited to, those derived from the following inorganic acids: hydrochloric, hydrobromic, hydroiodic, sulphuric, sulphurous, nitric, nitrous, phosphoric, and phosphorous. Examples of suitable organic anions include, but are not limited to, those derived from the following organic acids: acetic, propionic, succinic, glycolic, stearic, palmitic, lactic, malic, pamoic, tartaric, citric, gluconic, ascorbic, maleic, hydroxymaleic, phenylacetic, glutamic, aspartic, benzoic, cinnamic, pyruvic, salicyclic, sulfanilic, 2-acetyoxybenzoic, fumaric, phenylsulfonic, toluenesulfonic, methanesulfonic, ethanesulfonic, ethane disulfonic, oxalic, pantothenic, isethionic, valeric, lactobionic, and gluconic. Examples of suitable polymeric anions include, but are not limited to, those derived from the following polymeric acids: tannic acid, carboxymethyl cellulose.

It may be convenient or desirable to prepare, purify, and/or handle a corresponding solvate of the active compound. The term “solvate” is used herein in the conventional sense to refer to a complex of solute (e.g. active compound, salt of active compound) and solvent. If the solvent is water, the solvate may be conveniently referred to as a hydrate, for example, a mono-hydrate, a di-hydrate, a tri-hydrate, etc.

It may be convenient or desirable to prepare, purify, and/or handle the active compound in a chemically protected form. The term “chemically protected form”, as used herein, pertains to a compound in which one or more reactive functional groups are protected from undesirable chemical reactions, that is, are in the form of a protected or protecting group (also known as a masked or masking group or a blocked or blocking group). By protecting a reactive functional group, reactions involving other unprotected reactive functional groups can be performed, without affecting the protected group; the protecting group may be removed, usually in a subsequent step, without substantially affecting the remainder of the molecule. See, for example, Protective Groups in Organic Synthesis (T. Green and P. Wuts, Wiley, 1999).

For example, a hydroxy group may be protected as an ether (—OR) or an ester (—OC(═O)R), for example, as: a t butyl ether; a benzyl, benzhydryl (diphenylmethyl), or trityl (triphenylmethyl) ether; a trimethylsilyl or t-butyldimethylsilyl ether; or an acetyl ester (OC(═O)CH₃, OAc).

For example, an aldehyde or ketone group may be protected as an acetal or ketal, respectively, in which the carbonyl group (>C═O) is converted to a diether (>C(OR)₂), by reaction with, for example, a primary alcohol. The aldehyde or ketone group is readily regenerated by hydrolysis using a large excess of water in the presence of acid.

It may be convenient or desirable to prepare, purify, and/or handle the active compound in the form of a prodrug. The term “prodrug”, as used herein, pertains to a compound which, when metabolised (e.g. in vivo), yields the desired active compound. Typically, the prodrug is inactive, or less active than the active compound, but may provide advantageous handling, administration, or metabolic properties.

For example, some prodrugs are esters of the active compound (e.g. a physiologically acceptable metabolically labile ester). During metabolism, the ester group (—C(═O)OR) is cleaved to yield the active drug. Such esters may be formed by esterification, for example, of any of the carboxylic acid groups (—C(═O)OH) in the parent compound, with, where appropriate, prior protection of any other reactive groups present in the parent compound, followed by deprotection if required. Examples of such metabolically labile esters include those wherein R is C₁₋₇ alkyl (e.g. Me, Et); C₁₋₇ aminoalkyl (e.g. aminoethyl; 2-(N,N-diethylamino)ethyl; 2-(4 morpholino)ethyl); and acyloxy-C₁₋₇ alkyl (e.g. acyloxymethyl; acyloxyethyl; e.g. pivaloyloxymethyl; acetoxymethyl; 1-acetoxyethyl; 1-(1-methoxy-1-methyl)ethyl-carbonxyloxyethyl; 1-(benzoyloxy)ethyl; isopropoxy-carbonyloxymethyl; 1-isopropoxy-carbonyloxyethyl; cyclohexyl-carbonyloxymethyl; 1 cyclohexyl-carbonyloxyethyl; cyclohexyloxy-carbonyloxymethyl; 1-cyclohexyloxy-carbonyloxyethyl; (4-tetrahydropyranyloxy) carbonyloxymethyl; 1-(4-tetrahydropyranyloxyl)carbonyloxyethyl; (4-tetrahydropyranyl)carbonyloxymethyl; and 1-(4 tetrahydropyranyl)carbonyloxyethyl).

Also, some prodrugs are activated enzymatically to yield the active compound, or a compound which, upon further chemical reaction, yields the active compound. For example, the prodrug may be a sugar derivative or other glycoside conjugate, or may be an amino acid ester derivative.

A prodrug of a compound of formula (II) or (III) may include 3,4,5-trimethoxy-epicatechin-3-gallate.

Compounds, as described herein, may be in substantially purified form and/or in a form substantially free from contaminants. Each compound described herein may be isolated from a reaction mixture. Isolation refers to the separation of the product from unreacted starting material, other reaction products, reagents and, optionally, solvent.

The substantially purified form is at least 50% by weight, e.g., at least 60% by weight, e.g., at least 70% by weight, e.g., at least 80% by weight, e.g., at least 90% by weight, e.g., at least 95% by weight, e.g., at least 97% by weight, e.g., at least 98% by weight, e.g., at least 99% by weight.

Unless specified, the substantially purified form refers to the compound in any stereoisomeric or enantiomeric form. For example, in some embodiments, the substantially purified form refers to a mixture of stereoisomers, i.e., purified with respect to other compounds. In other embodiments, the substantially purified form refers to one stereoisomer, e.g., optically pure stereoisomer. In some embodiments, the substantially purified form refers to a mixture of enantiomers, for example the substantially purified form may refer to an equimolar mixture of enantiomers (i.e., a racemic mixture, a racemate). In other embodiments, the substantially purified form refers to one enantiomer, e.g. optically pure enantiomer.

In some embodiments, the contaminants represent no more than 50% by weight, e.g., no more than 40% by weight, e.g., no more than 30% by weight, e.g., no more than 20% by weight, e.g., no more than 10% by weight, e.g., no more than 5% by weight, e.g., no more than 3% by weight, e.g., no more than 2% by weight, e.g., no more than 1% by weight.

The purity may be established by one or more of analytical and spectroscopic techniques including NMR (e.g. ¹³C or ¹H), LC-MS, HPLC, TLC, UV, IR and gravimetric analysis.

Unless specified, the contaminants refer to other compounds, that is, other than stereoisomers or enantiomers. In some embodiments, the contaminants refer to other compounds and other stereoisomers. In some embodiments, the contaminants refer to other compounds and the other enantiomer.

The substantially purified form may be at least 60% optically pure (i.e., 60% of the compound, on a molar basis, is the desired stereoisomer or enantiomer, and 40% is the undesired stereoisomer or enantiomer), e.g., at least 70% optically pure, e.g., at least 80% optically pure, e.g., at least 90% optically pure, e.g., at least 95% optically pure, e.g., at least 97% optically pure, e.g., at least 98% optically pure, e.g., at least 99% optically pure.

Techniques for the separation of the compounds include, where appropriate, chromatography, including flash column chromatography, preparative HPLC and preparative TLC, crystallisation, distillation, and aqueous-organic extraction amongst others.

Each and every compatible combination of the embodiments described above is explicitly disclosed herein, as if each and every combination was individually and explicitly recited.

Various further aspects and embodiments of the present invention will be apparent to those skilled in the art in view of the present disclosure.

“and/or” where used herein is to be taken as specific disclosure of each of the two specified features or components with or without the other. For example “A and/or B” is to be taken as specific disclosure of each of (i) A, (ii) B and (iii) A and B, just as if each is set out individually herein.

Unless context dictates otherwise, the descriptions and definitions of the features set out above are not limited to any particular aspect or embodiment of the invention and apply equally to all aspects and embodiments which are described.

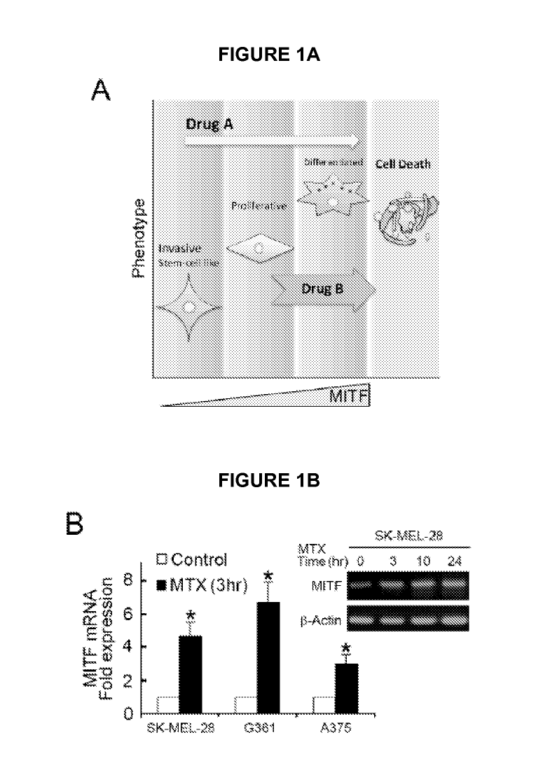

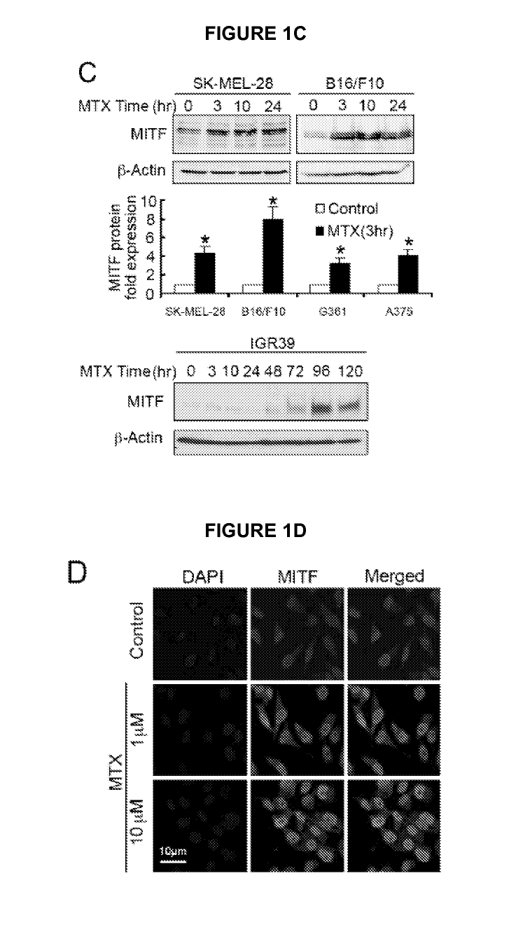

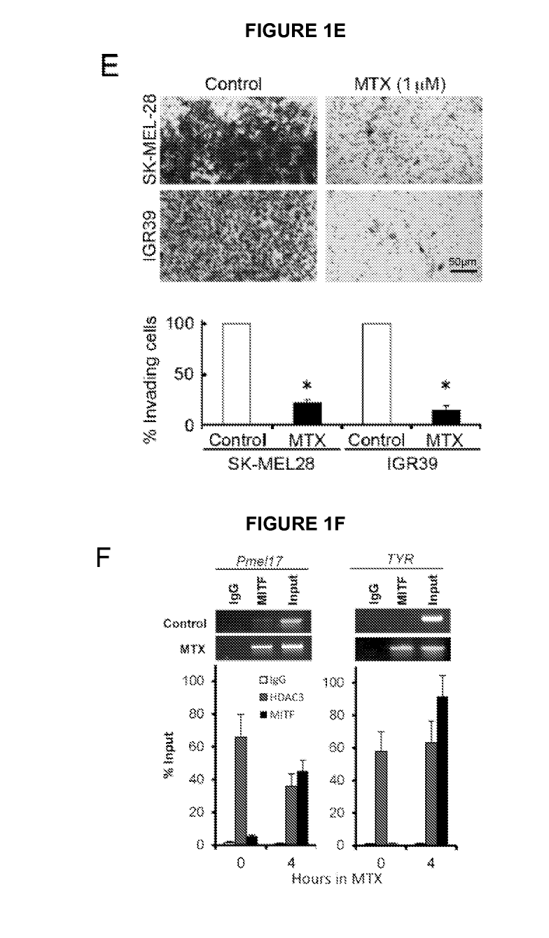

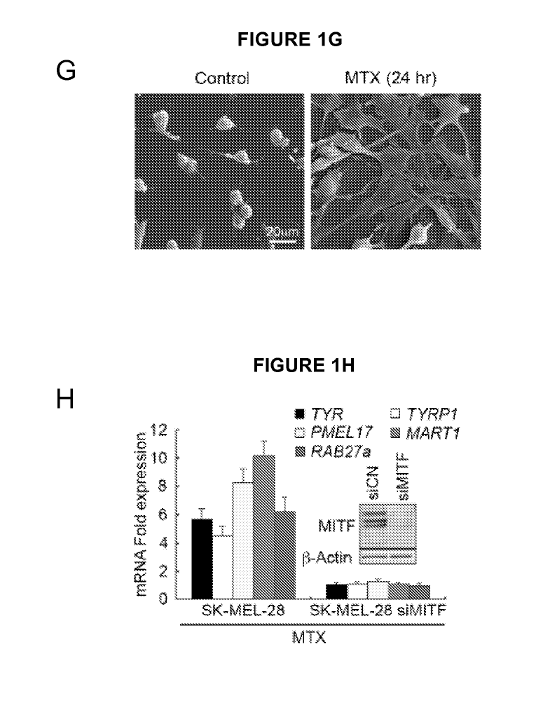

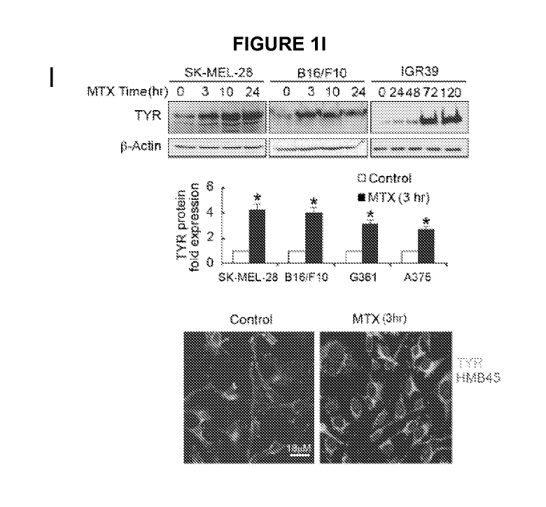

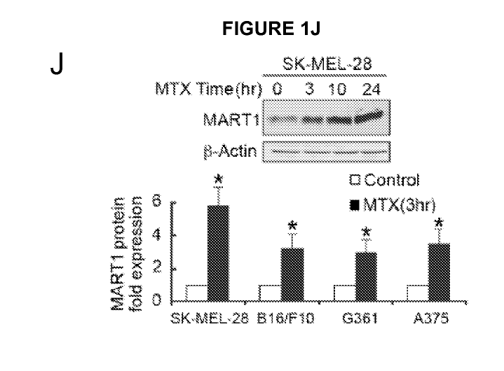

The Microphthalmia-associated transcription factor gene MITF7 has been termed a lineage-addiction oncogene and is key regulator of melanoma biology (Garraway). Mutations affecting MITF function are linked to melanoma predisposition (Bertolotto; Yokoyama). MITF acts as a rheostat (FIG. 1A) that determines sub-population identity in response to microenvironmental cues (Carreira 2006; Hoek; Cheli 2011). Low MITF expression, for example in response to hypoxia, leads to G1 arrest. Invasive cells with stem-like properties that are able to initiate tumors with high efficiency also express low MITF (Carreira 2006; Carreira 2011). By contrast, elevated MITF activity leads either to differentiation or proliferation, most likely depending on MITF post-translational modifications (Carreira 2005; Loercher; Cheli 2010).

To eradicate melanomas, it is important that the invasive cell population that contributes to metastasis, and to renewal of the tumor population, is eliminated. Stem cell-like melanoma cells are highly invasive and have the potential to propagate and to replenish the tumor cell population. One possible treatment strategy is to drive the differentiation of these cell-like melanoma cells into a differentiated melanoma cell that will be highly susceptible to melanoma specific drugs.

A two-step therapeutic approach (FIG. 1A) has been developed to circumvent many problems associated to both genetic and phenotypic heterogeneity: First, elevate MITF expression to eradicate invasive stem-like cells; then, use the MITF-induced melanocyte-specific enzyme tyrosinase to activate a pro-drug able to target an enzyme critical to cell viability in a cell type-specific fashion.

Methotrexate (MTX) was identified as an active agent capable of elevating MITF levels. MTX is a differentiating agent in widespread clinical use. It is a slow-tight binding competitive inhibitor of dihydrofolate reductase (DHFR), as an effective activator of MITF expression. MTX increased both MITF mRNA (FIG. 1B) and protein (FIGS. 1C and 1D) in both mouse (B16/F10) and human (SK-MEL-28, G361, A375) cell lines, consistent with previous observations that MTX can increase melanogenesis and accelerate melanosome export (Sánchez-del-Campo Pigment Cell Melanoma Res 2009). MTX also upregulated MITF expression in the amelanotic and highly invasive melanoma cell line IGR39 (FIG. 1C, lower panel). Importantly, and consistent with the MITF rheostat model (FIG. 1A), MTX also eliminated invasiveness of both SK-MEL-28 and IGR39 cells in a matrigel Boyden chamber assay (FIG. 1E). The reduction in invasiveness on MTX addition was mediated by increased MITF expression, as siRNA-mediated depletion of MITF reversed the effect of MTX.

Chromatin immunoprecipitation (ChIP) assays confirmed that the MTX increased binding of MITF to its target genes tyrosinase (TYR) and Pmel17 (FIG. 1F; HDAC3 is used as control), and also induced a dendritic cell morphology characteristic of MITF-driven differentiation (FIG. 1G), and the first observable parameter of melanoma cell differentiation (Carreira et al. 2005; Tachibana et al.; Serafino et al.). Consistent with these data, MTX increased mRNA expression of the MITF differentiation targets TYR, Pmel17, Rab27a, TYRP1 and MART117 that was prevented by MITF-specific siRNA (FIG. 1H). MTX-driven differentiation was also reflected in increased protein expression of both TYR (FIG. 1I) in multiple melanoma cell lines, including the amelanotic cell line IGR39, as detected by either western blot or immunofluorescence and MART1 (FIG. 1J).

Compared with untreated SK-MEL-28 cells, MTX substantially increased the occupancy of MITF on the TYR promoter (from 1.5% in untreated cells to 25.2% in MTX-treated cells with respect to an input control) and on the promoter/enhancer of the Pmel17 gene (from 5.4% in untreated cells to 45.4% in MTXtreated cells with respect to an input control). No binding was observed to control regions lacking MITF-target sites.

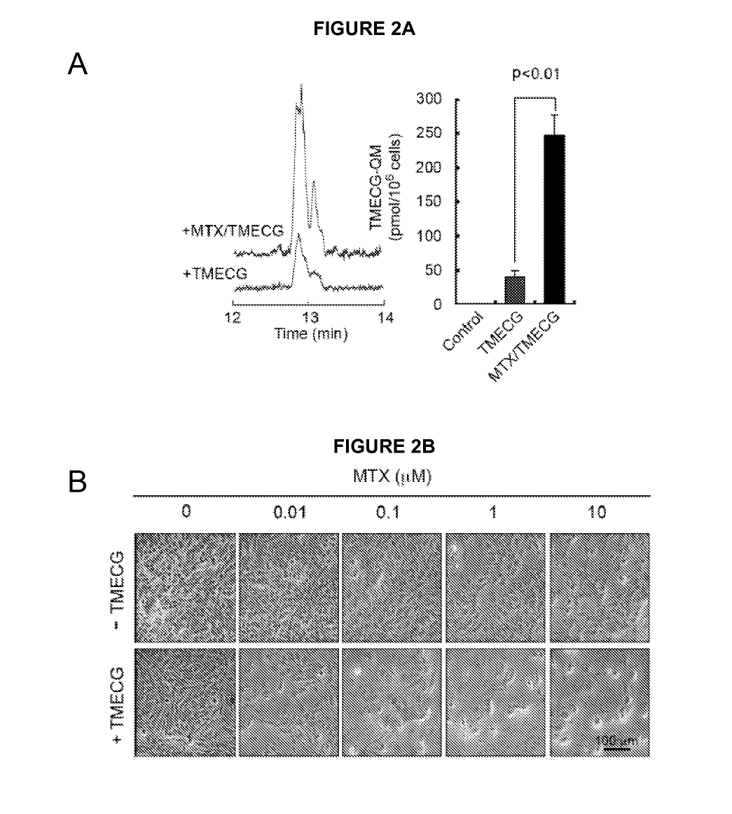

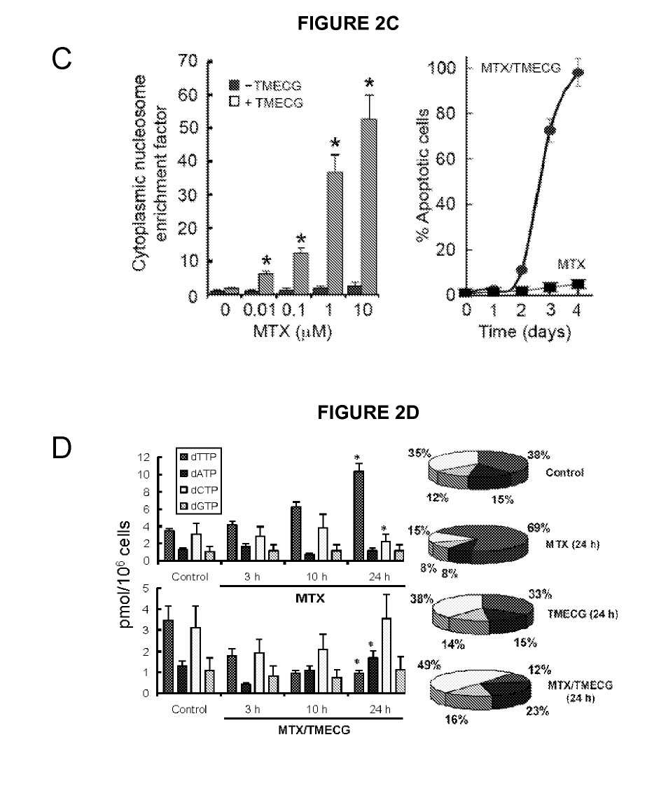

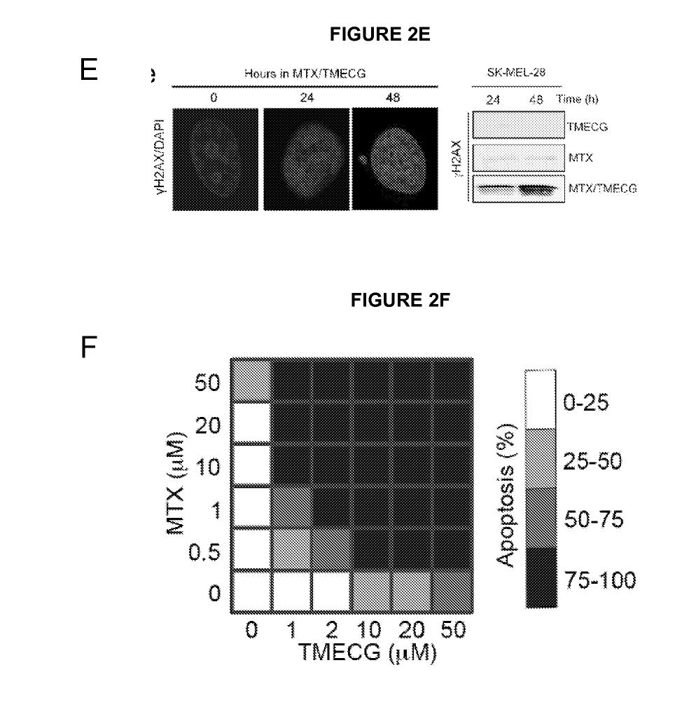

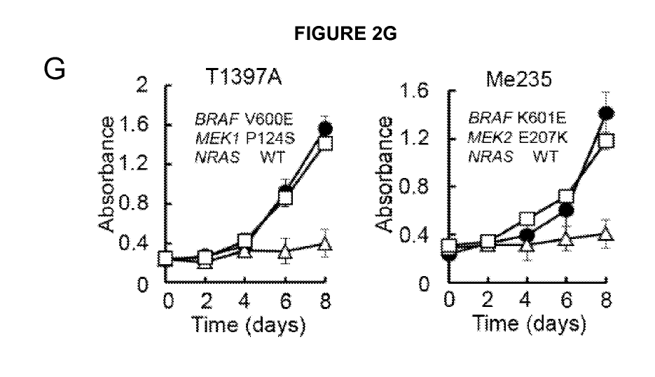

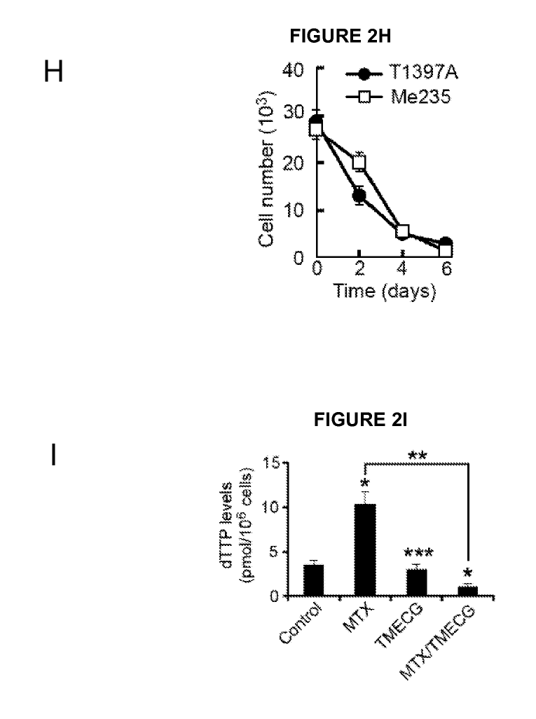

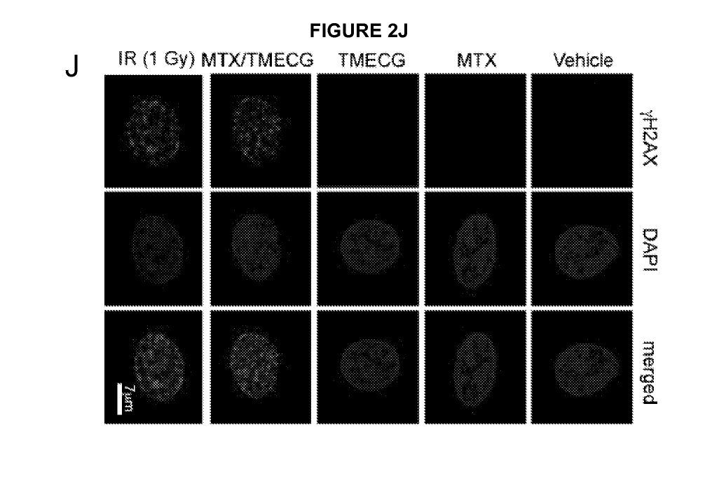

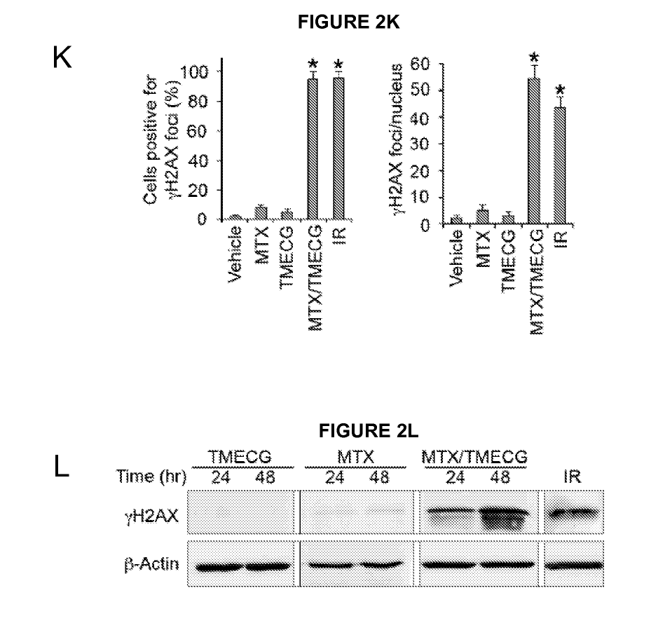

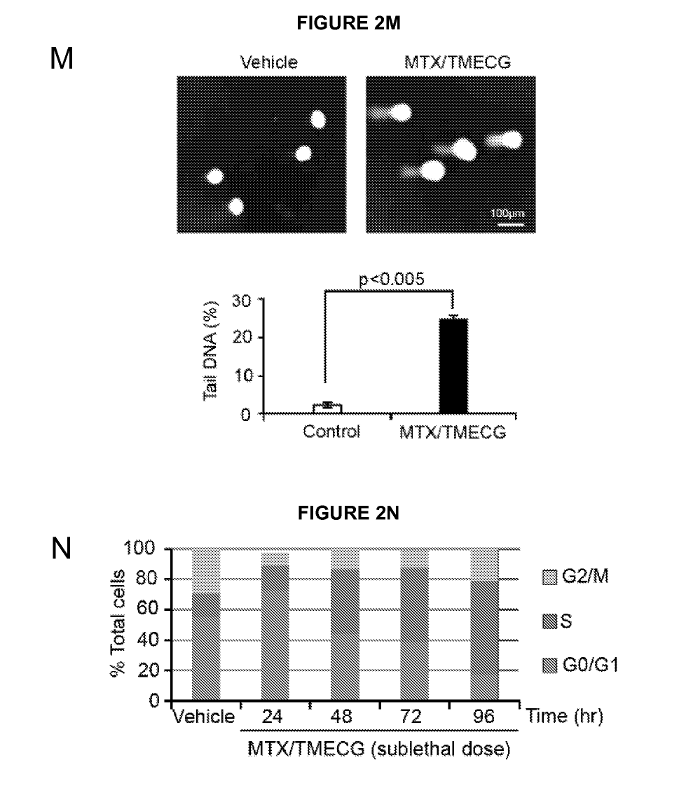

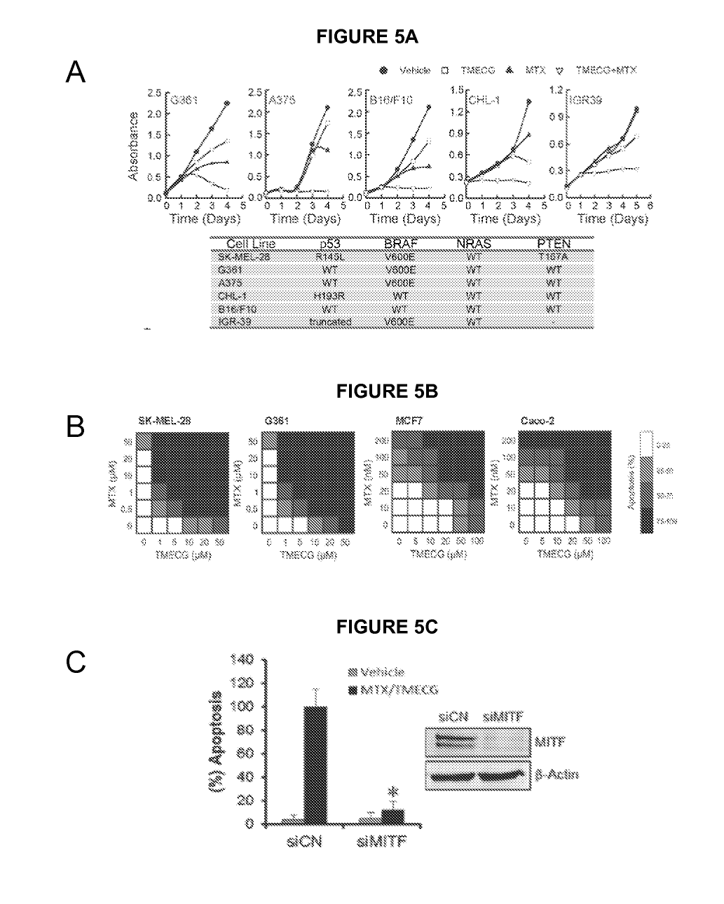

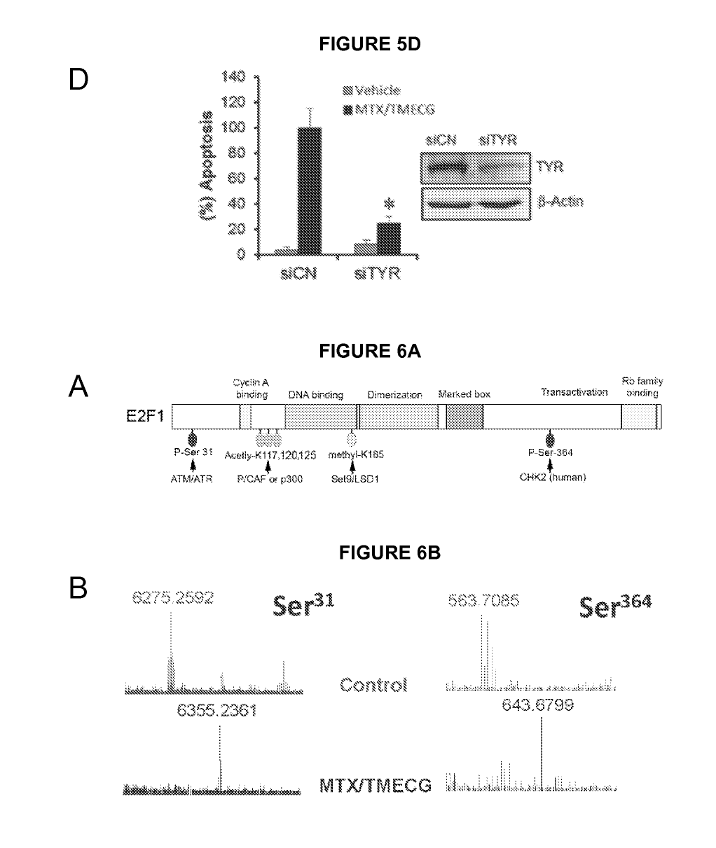

The increased TYR expression in response to MTX-mediated MITF activation was identified by the present inventors as an opportunity to implement a second arm of a two-step strategy. 3-O-(3,4,5-trimethoxybenzoyl)-(−)-epicatechin (TMECG) is an anti-folate pro-drug designed to be activated by tyrosinase (Sanchez-del-Campo Mol Pharm 2009), in effect generating a cell-type-specific cytotoxic agent. HPLCMS/MS experiments confirmed that MTX-induced TYR overexpression greatly contributed to the activation of the pro-drug TMECG to its corresponding quinone methide (TMECG-QM) (FIG. 2A and Table 1) that acts as a potent competitive inhibitor of DHFR for dihydrofolate (Sanchez-del-Campo Mol Pharm 2009). Treatment of SK-MEL-28 cells with MTX alone reduced proliferation but did not induce apoptosis (FIG. 2B, upper panels), whereas MTX at concentrations as low as 10 nM in combination with TMECG led to substantial apoptosis (FIG. 2B lower panels and FIG. 2C) and induced MITF expression (FIG. 5A). The combination of a single dose of MTX and TMECG was also highly effective, with apoptosis occurring in close to 100% of cells after 4 days treatment (FIG. 2C) and titration of both compounds demonstrated they acted synergistically (FIG. 2F). Notably, the MTX/TMECG also prevents proliferation melanoma cell lines independently of the mutational status of genes such as p53, BRAF, NRAS or PTEN and was also effective in the amelanotic melanoma cell line IGR39 (FIG. 5A).

It was therefore surmised that MTX/TMECG could also be effective against BRAF-mutant melanomas that have developed genetic resistance to BRAF and/or MEK inhibitors. MTX/TMECG was tested against two low passage melanoma cell lines (fewer than 10 passages) derived from patients with activating BRAF mutations that are resistant to both BRAF-inhibitor and MEK-inhibitor therapy, owing to the presence of activating mutations in MEK1 or MEK2 (Nikolaev et al., 2011). In the MTT assays (FIGS. 2G and 5A) a low starting number of cells (2×10³) was used, so that any proliferation could be readily visualized. As expected, the PLX-4720 inhibitor of activated BRAF failed to impact significantly on proliferation (FIG. 2G). By contrast, the MTX/TMECG combination therapy was highly effective. By starting with 3×10⁴ cells and counting cell numbers the effectiveness of MTX/TMECG in inducing cell death in these drug-resistant cells was readily apparent (FIG. 2H). Similar effects were seen on melanoma cells derived from dissociated fresh melanoma metastases isolated directly from patients (images not shown).

To distinguish between melanoma and non-melanoma cells in the freshly-dissociated tumors, a DOPACHROME TAUTOMERASE (DCT) promoter-mCherry reporter virus was used, which expresses mCherry only in the melanocyte lineage. In this case, MTX/TMECG treatment for 6 days led to a 5-fold reduction in mCherry-positive cells indicating effectiveness in cells directly isolated from patient-derived tissue.

To verify that MTX/TMECG synergy is cell type-specific, titration experiments were performed in melanoma (SK-MEL-28, G361), breast (MCF7) and colon (Caco-2) cancer cell lines (FIG. 5B). As expected the melanoma cell lines were substantially less sensitive to MTX alone than the non-melanoma lines. The sensitivity to TMECG alone was between 2 and 5-fold greater in the melanoma cell lines since both melanoma cell lines express low levels of TYR. However, MTX synergistically increased the sensitivity of the melanoma cells to TMECG, presumably by up-regulating MITF and TYR, while in the non-melanoma cell lines the effects were at best additive. siRNA-mediated depletion of MITF (FIG. 5C), or TYR (FIG. 5D), substantially reduced cell death and confirmed their key requirement for the effectiveness of the MTX/TMECG drug combination. Thus the synergy between the two compounds appears limited to TYR-positive melanoma cells, a key aim in the design of a cell type specific anti-melanoma therapy.

DHFR activity is critical for thymidine synthesis. Contrary to the effects of MTX in most cancer cells (Wang et al.), this drug increased dTTP levels in melanoma, generating a thymidine excess (FIG. 2D, upper panel). This paradoxical response of melanoma cells to a cytotoxic drug that typically depletes dTTP levels may be explained by the fact that DHFR is a direct target for MITF (Strub et al.). However, MTX and TMECG combined generated a nucleotide imbalance that strongly favoured dTTP depletion (FIG. 2D, lower panel). In melanoma, MTX alone leads to increased dTTP levels, whereas dTTP was significantly reduced by the MTX/TMECG combination (FIG. 2I), leading to a nucleotide imbalance (FIG. 2D). Consistent with the effects of combination treatment being mediated via thymidine depletion, combined MTX/TMECG treatment of SK-MEL-28 cells, but not MTX or TMECG alone, led to massive DNA damage in melanoma cells indicated by the accumulation of γH2AX (FIG. 2E).

The ability of MTX to elevate dTTP in melanoma, but not other cancer types may be partly explained by the fact that DHFR is regulated by MITF (Strub et al; data not shown) which is strongly up-regulated by MTX. Moreover, because TMECG-QM acts as a competitive inhibitor of DHFR with respect to DHF, the observed MTX-dependent depletion of this substrate (Table 1) could explain the high synergy observed upon co-treatment with MTX and TMECG in melanoma cells (FIGS. 5A and 5B) where TYR converts TMECG to its quinone methide (FIG. 2A).

Thymidine depletion induces DNA double-strand break (DSB) formation (Pardee et al.) characterized by phosphorylation of histone H2AX at Ser139 (γH2AX) by ATM/ATR kinases and the subsequent rapid formation of γH2AX foci at the DSB sites (Kinner et al.). Consistent with the effects of combination treatment being mediated via thymidine depletion, immunofluorescence revealed that combined MTX/TMECG treatment of SKMEL-28 cells, but not MTX or TMECG alone, led to accumulation of γH2AX foci by 48 hr (FIGS. 2J and 2K), a result confirmed by western blotting (FIG. 2L). The increase in γH2AX foci was accompanied by the induction of DSBs as determined using a comet assay (FIG. 2M). Moreover, consistent with the MTX/TMECG combination causing S-phase associated DNA damage, sub-lethal doses of MTX/TMECG coupled with flow cytometry revealed accumulation of cells in S-phase (FIG. 2N).

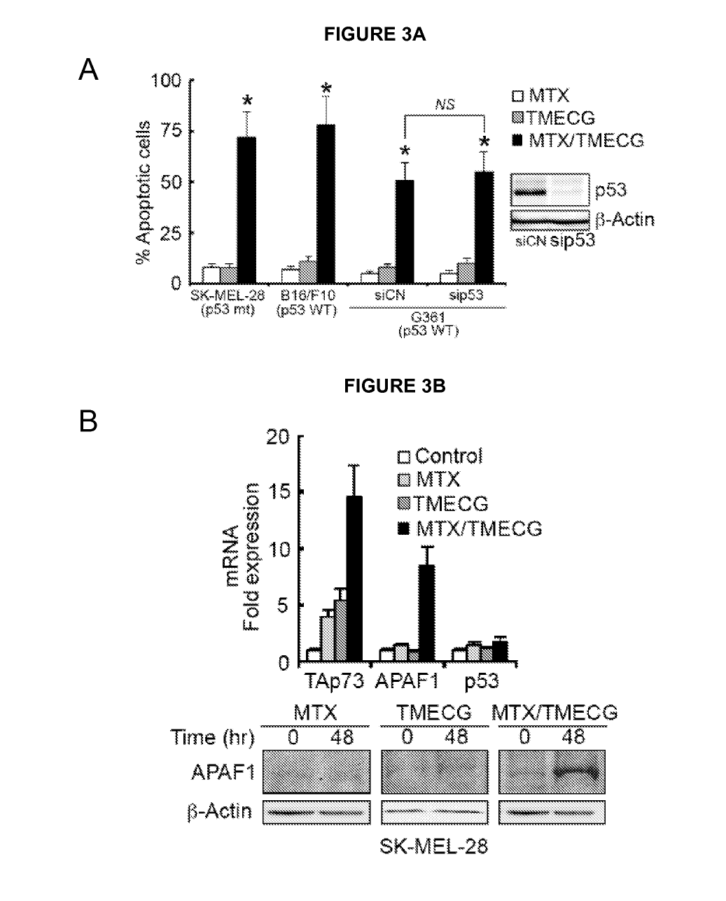

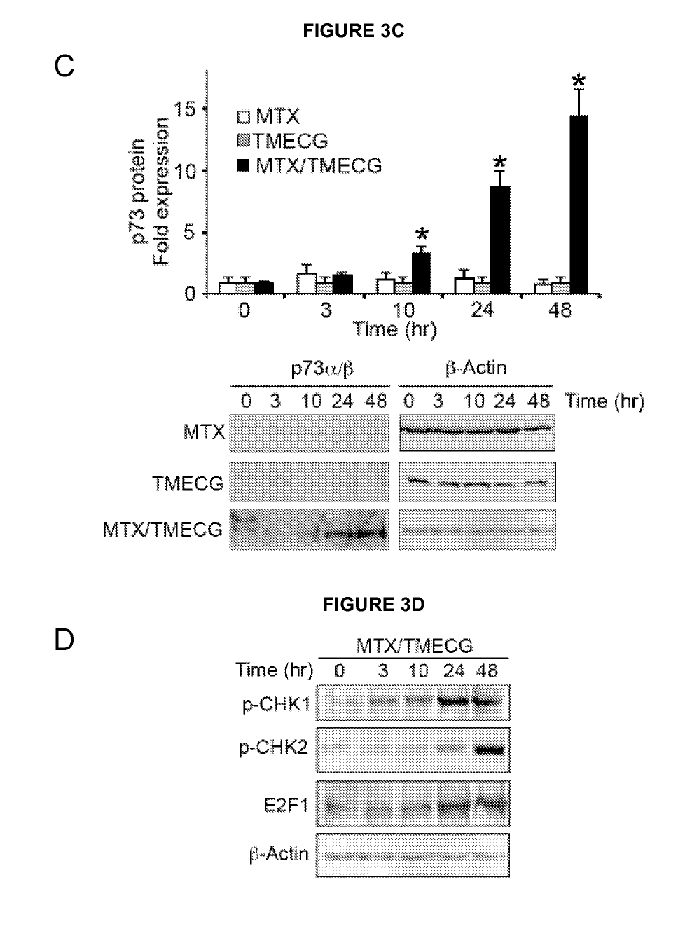

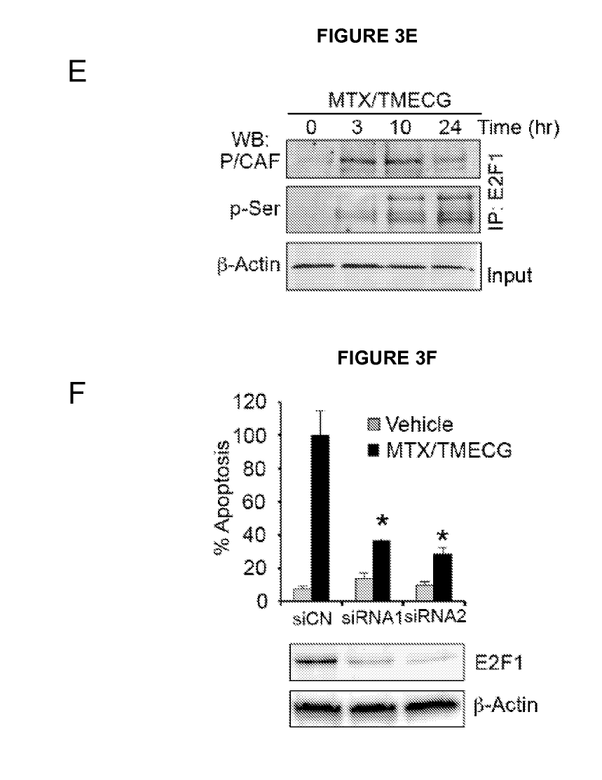

Although p53 is usually WT in melanoma (Box et al.), apoptosis triggered by MTX/TMECG treatment was independent of p53 mutation status (FIG. 3A) and was not affected by p53 silencing in G361 cells (FIG. 3A). Although p53 mRNA levels in SKMEL-28 cells were unaffected by the MTX/TMECG combination (FIG. 3B), MTX and TMECG combined, dramatically induced the mRNA (FIG. 3B) and protein expression (FIG. 3C) of the pro-apoptotic transactivating form of p73 (TAp73) that was accompanied by elevated expression of the apoptosis protease-activating factor 1 (Apaf1) (FIG. 3B, right panel).

p73 expression is controlled by E2F1 (Dobbelstein), which in turn is stabilized by phosphorylation by Chk2 at Ser364, ATM kinase at Ser31, or acetylation by P/CAF at lysines 117, 120, and 125 (Urist; Lin; Martinez-Balbas). DNA-damage induced by the MTX/TMECG combination led to increased Chk1 and Chk2 phosphorylation and increased E2F1 protein (FIG. 3D). Immunoprecipitation of E2F1 revealed that MTX/TMECG increased its phosphorylation and association with the P/CAF acetyl transferase (FIG. 3E).

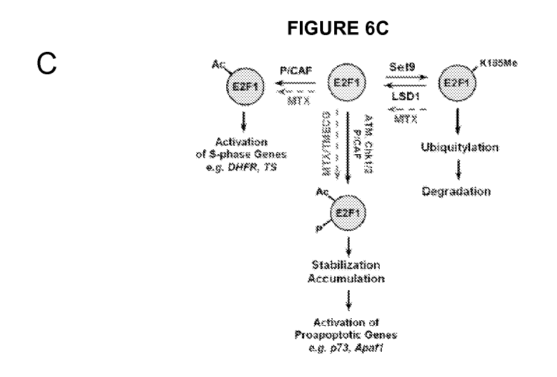

Mass spectrometry analysis of immunoprecipitated E2F1 confirmed that MTX/TMECG increased both phosphorylation (FIG. 6) and acetylation of E2F1, and revealed loss of methylation of E2F1 at Lys185 (Table 2), a modification that inhibits acetylation and promotes E2F1-degradation and prevents stabilization of E2F1 in response to DNA damage (Kontaki). siRNA-mediated silencing of E2F1 (FIG. 5F) significantly decreased the sensitivity of SK-MEL-28 cells to MTX/TMECG-induced apoptosis compared to a control siRNA (siCN). Although it cannot be ruled out that E2F1-depletion blocks apoptosis by preventing passage to S-phase, collectively the data are consistent with a mechanism by which manipulating MITF, and consequently TYR levels, via MTX treatment renders melanoma cells sensitive to TMECG-induced depletion of dTTP pools and p53-independent and E2F1-driven apoptosis.

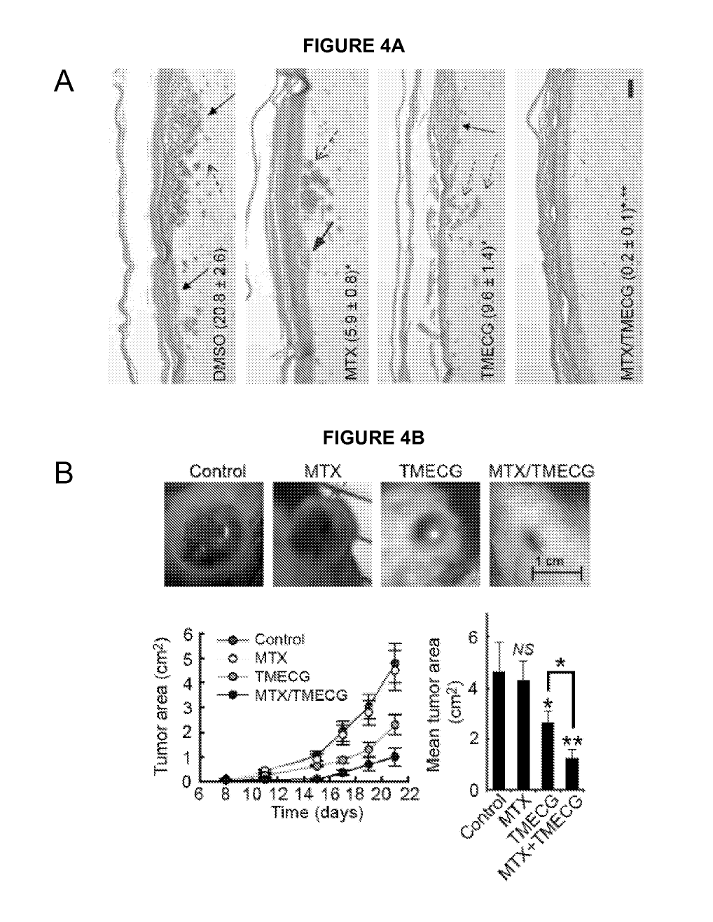

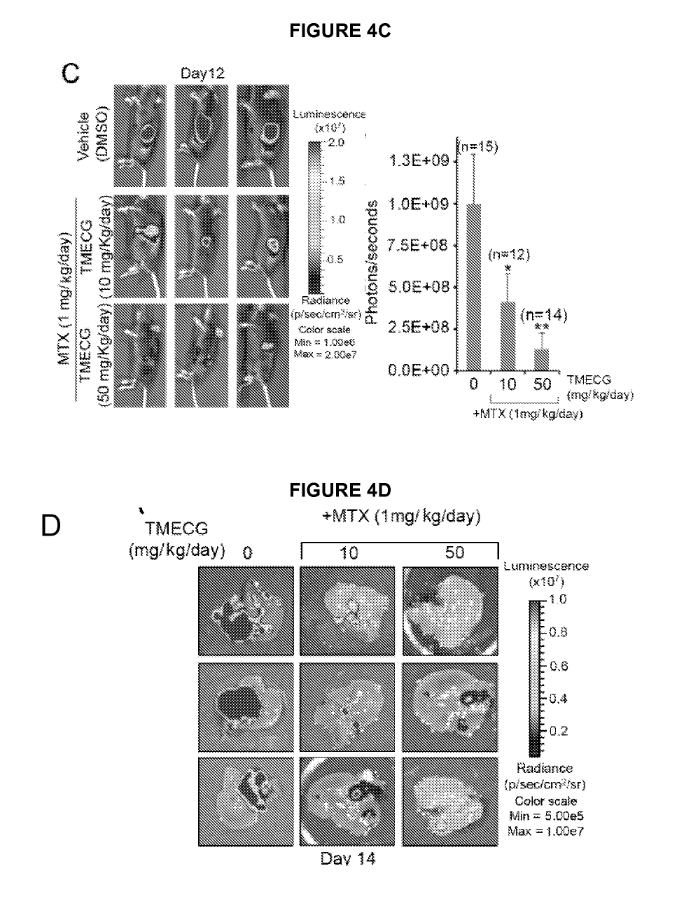

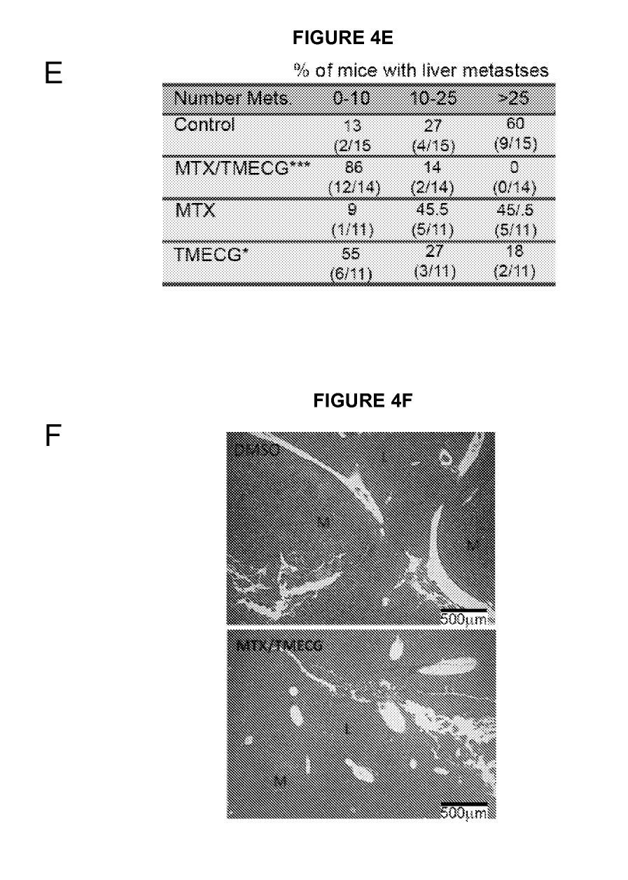

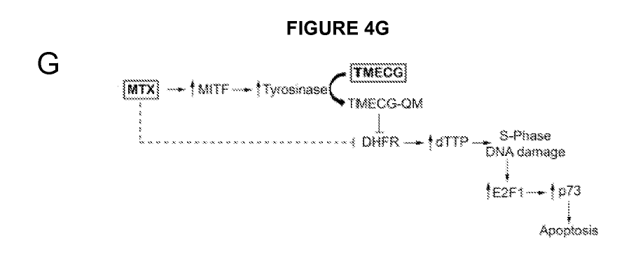

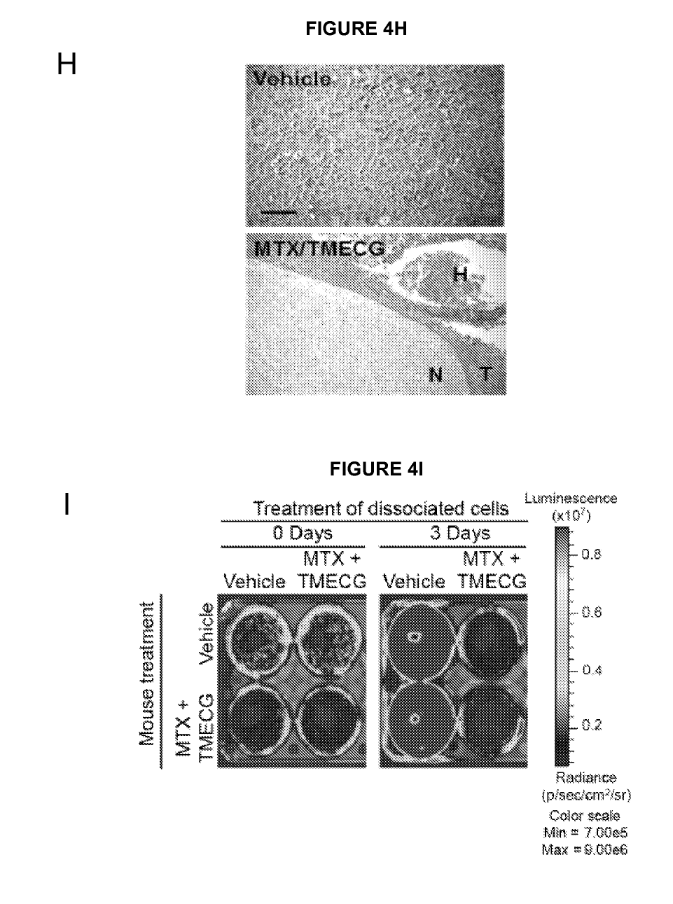

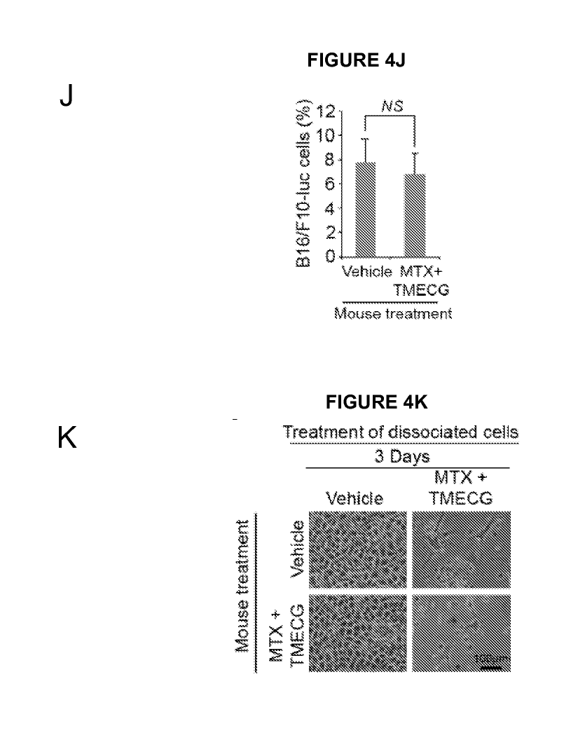

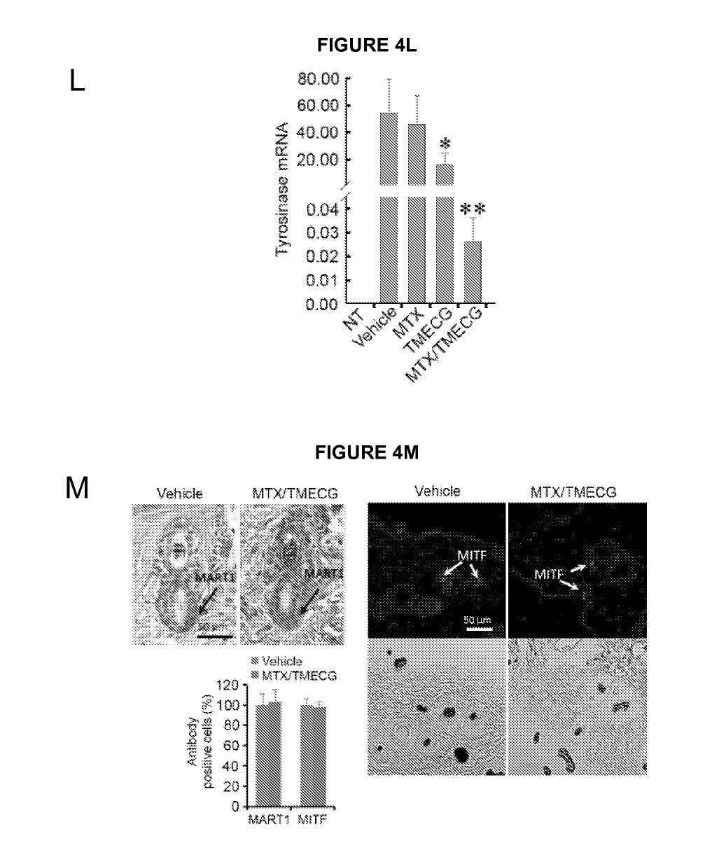

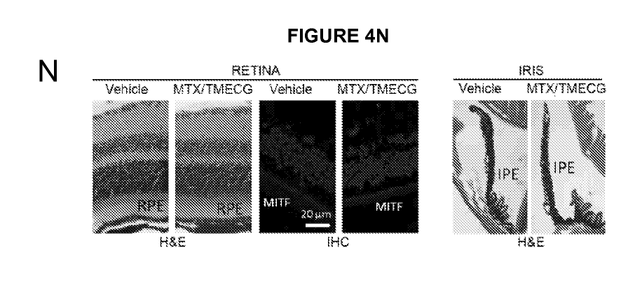

The profound effects of the MTX/TMECG combination in vitro led to testing the antitumorigenic efficacy of the combination in vivo. In a reconstituted skin model of melanoma, in which melanocytes are replaced by A375 melanoma cells (FIG. 4A), massive melanoma nodes were observed within the epidermis 21 days after seeding, and evidence of early metastasis into dermal structures was observed in untreated skin. In contrast to individual treatment with either MTX or TMECG, 3D cultures were mostly free of melanoma cells after 14 days of treatment with the MTX/TMECG combination. In an independent approach, B16/F10 melanoma cells were injected subcutaneously into C57BL/6 mice, a syngeneic melanoma model in which the host mice retain an intact immune system that plays a major role in the evolution of human melanoma (Zaidi). Visual examination revealed that compared to untreated mice, tumor growth was significantly reduced by TMECG treatment, but not by MTX treatment (FIG. 4B). Tumors extracted from MTXtreated mice were softer, easy to dissociate and more melanized than those obtained for vehicle-treated mice, consistent with MTX-induced expression of MITF and TYR activity.

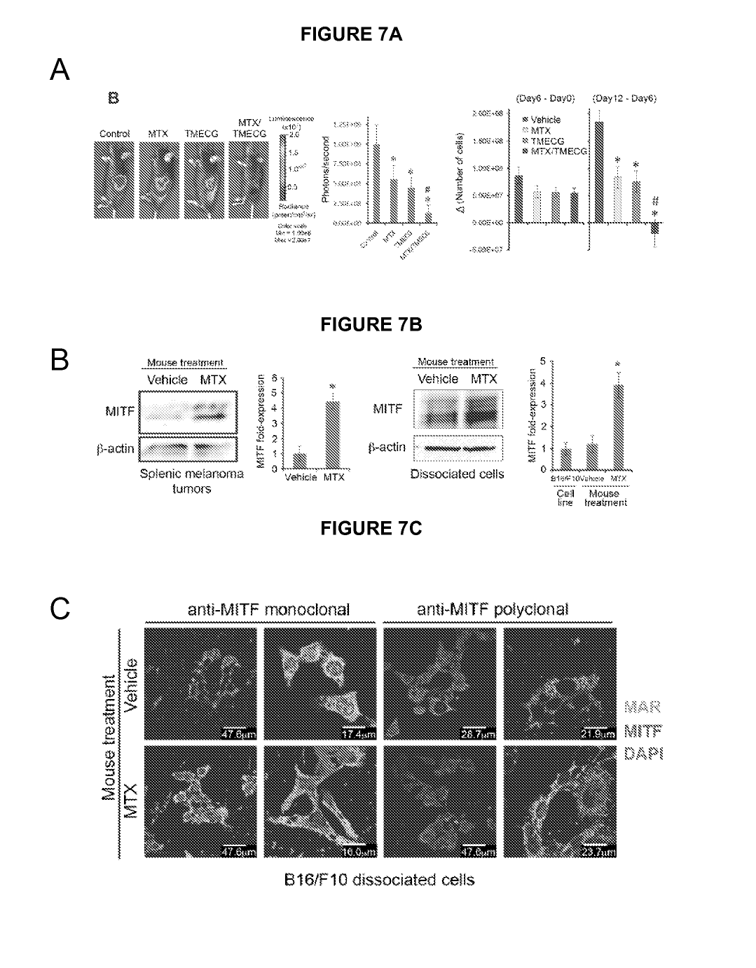

As anticipated, the combination of MTX and TMECG acted synergistically to inhibit tumor growth. Using B16 melanoma cells expressing a luciferase reporter (FIG. 4C), quantification of the in vivo luminescence signal confirmed that the MTX/TMECG combination was highly effective at reducing tumor burden. Whereas MTX or TMECG alone had around 2-fold effect, a synergistic reduction in luciferase was seen using the combination (FIG. 7A, left panels). Importantly, between day 6 and day 12, MTX or TMECG alone led to decreased numbers of melanoma cells in the tumors compared to vehicle treated mice, however, by day 12 the MTX/TMECG combination had reduced the number of melanoma cells within the tumors compared to day 6, an indication of an effective therapeutic response. B16/F10 tumors treated with DMSO showed their usual histological appearance of poor differentiation and limited necrosis (FIG. 4H upper panel). In contrast, 14 days treatment with MTX/TMECG induced obvious haemorrhagic necrosis, with necrotic areas of approximately 75% (FIG. 4H, lower panels). Necrosis in splenic tumors was less evident when mice were treated with MTX or TMECG alone (4%±2%; and 11%±3%, respectively; data not shown). Consistent with the results obtained in cultured melanoma cells, MTX effectively induced MITF expression in mice as determined by western blotting of tumors in vivo (FIG. 7B, left panel) or western blotting or immunofluorescence of dissociated tumor cells (FIGS. 7B, right panel, and 7C, respectively).

Significantly any residual cells surviving MTX/TMECG treatment in vivo retained their sensitivity to the drug combination. Dissociated luciferase-tagged B16/F10 tumor cells from vehicle or MTX/TMECG-treated mice were assayed for luciferase activity immediately after plating or three days later. Cells from both vehicle and MTX/TMECG-treated animals were able to proliferate in culture in the absence of drug, but treatment with MTX/TMECG retained its efficacy, reducing luciferase activity and cell number up to 18-fold, irrespective of whether they were derived from control or MTX/TMECG-treated mice (FIGS. 4I-4K). Thus any cells in vivo surviving MTX/TMECG treatment do not appear to acquire genetic or phenotypic resistance to the drug combination.

Since elevated MITF expression in response to MTX inhibits invasiveness (FIG. 1E), it was investigated whether MTX/TMECG administration after injection of melanoma cells could prevent melanoma dissemination from the spleen to the liver, one of the preferential metastatic locations for melanomas. Luciferase-tagged B16 cells were injected into the spleens of C57BL/6 mice and after 14 days treatment, tumor expansion was measured (FIG. 4D). Luciferase imaging showed that MTX/TMECG treated mice had a substantially lower burden of macroscopic liver metastases, with no mice bearing >25 macroscopic liver metastases compared with controls (FIG. 4E). Histological analysis of livers (FIG. 4F) revealed that when calculated as the percentage of liver volume, metastatic volume was 55±12% for vehicle and 6±2% for MTX/TMECG-treated mice (P=0.002). These data were also confirmed by real-time RT-PCR analysis designed to detect melanoma specific TYR mRNA in mouse livers (FIG. 4L). Thus, the MTX/TMECG combination leads to a dramatic inhibition of melanoma growth both in vitro and in vivo.

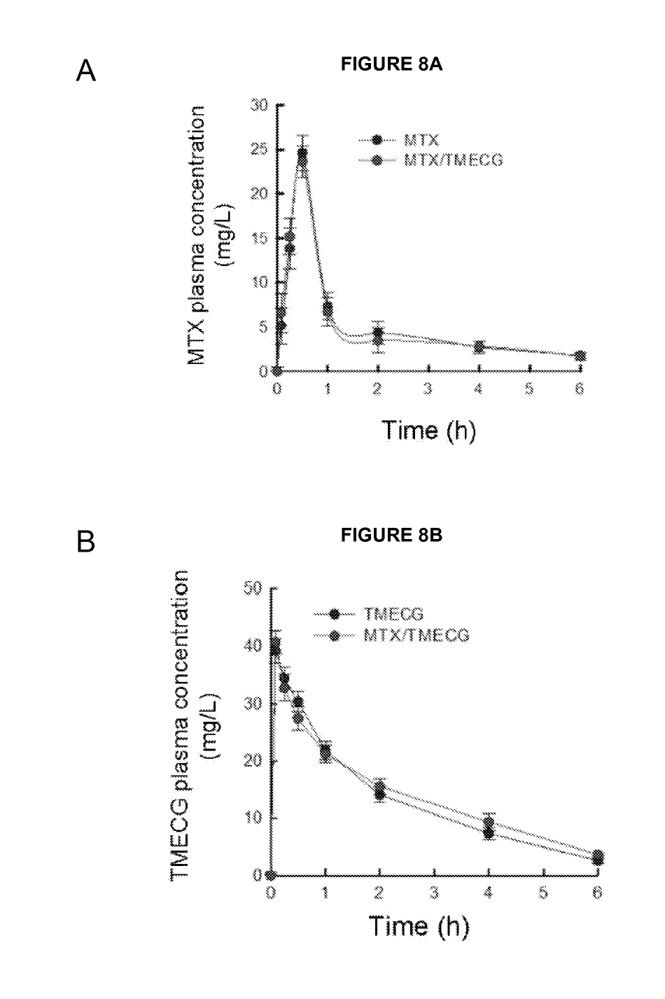

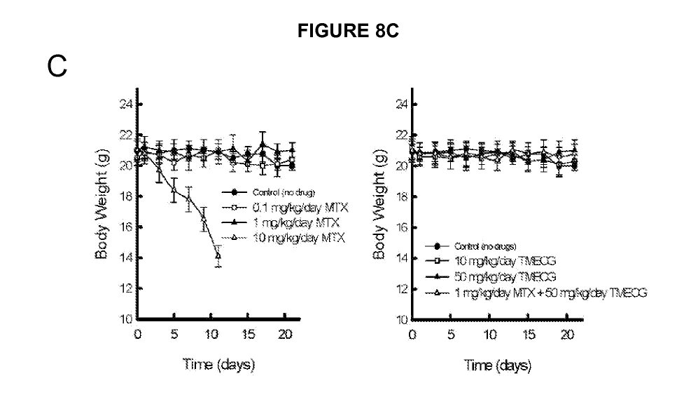

The potential toxicity of the MTX/TMECG combination was tested in mice. After administration, neither MTX nor TMECG affected the levels or clearance of the other compound in plasma (FIGS. 8A and 8B, respectively, and Table 3), and although high doses of MTX (10 mg/Kg/day) induced some weight loss in mice as expected, the doses of MTX used to treat melanoma in this study alone (1 mg/Kg/day) or in combination with up to 50 mg/Kg/day TMECG had no effect on mouse weight (FIG. 8C). Moreover, no obvious deleterious effect of the MTX or TMECG combination was evident on non-melanoma TYR and MITF-positive cells such as skin melanocytes (FIG. 4M) or the pigmented eye epithelia of the retina (RPE) and iris (IPE) (FIG. 4N) presumably because unlike melanoma, these TYR/MITF-positive cells are not proliferating and are therefore insensitive to DHFR inhibition and dTTP-depletion.

Collectively, the data indicate that MTX/TMECG combination therapy used here (FIG. 4G) is highly effective and has several key advantages compared to more conventional strategies.

Certain aspects and embodiments of the invention will now be illustrated by way of example and with reference to the figures described above.

Although the present invention and its advantages have been described in detail, it should be understood that various changes, substitutions and alterations can be made herein without departing from the spirit and scope of the invention as defined in the appended claims.

The present invention will be further illustrated in the following Examples which are given for illustration purposes only and are not intended to limit the invention in any way.

### Experimental and Results

### General Experimental

Immunofluorescence and western blotting were performed using standard protocols and commercially available antibodies. Proliferation was measured using MTT assays. TMECG was synthesized from catechin as described (Sanchez-del-Campo, L. et al. J. Med. Chem. 2008). For tumor formation assays B16 F10 (5.0×10⁵) melanoma cells were injected subcutaneously or intra-splenically (3.0×10⁵) of C57/B16 mice and tumors examined visually or using the IVIS Imaging System. Reconstituted skin was obtained from MaTek Corp. ChIP assays were performed using the Magna ChIP™ G kit and appropriate antibodies. dNTP pools were assayed as described (Angus).

### Reagents and Antibodies

TMECG was synthesized from catechin29 MTX was obtained from Sigma (Madrid, Spain). Antibodies against the following proteins were used: β-Actin (Sigma; Monoclonal clone AC-15), Apaf1 (BD Biosciences, Sparks, Md.; Polyclonal), phospho-Chk1 (Ser345) (Cell Signaling Tech., Danvers, Mass.; Monoclonal clone 133D3), phospho-Chk2 (Thr68) (Millipore; Madrid, Spain; Monoclonal clone E126), phospho-H2A.X (Ser139) (Millipore; Monoclonal clone JBW301), HDAC3 (Millipore, Monoclonal clone 3G6), E2F1 (Millipore; Monoclonal clones KH20 and KH95), MART1 (Sigma; Monoclonal clone A103), MITF (Millipore; Monoclonal clone C5), p53 (Santa Cruz Biotechnology; Monoclonal clone DO-1), p73 (a and 13) (Millipore; Polyclonal), P/CAF (Abcam, Cambridge, UK; Polyclonal), Pmel17 (Dako Inc., Carpinteria, Calif.; Monoclonal clone HMB45), phospho-Ser (Sigma; Monoclonal clone PSR-45), and TYR (Santa Cruz Biotechnology; Polyclonal).

### Cell Lines, Proliferation and Apoptosis Assays

Melanoma cell lines of human and mouse origin were obtained from ATCC and maintained in the appropriate culture medium supplemented with 10% FBS and antibiotics. Cell viability was evaluated using the 3-(4,5-dimethylthiazol-2-yl)-2,5-diphenyltetrazolium bromide (MTT) cell proliferation assay.

The induction of apoptosis was assessed by performing cytoplasmic histone-associated DNA fragmentation using a kit from Roche Diagnostics (Barcelona, Spain). An ELISA assay was used to detect that detects of mono- and oligonucleosomes in the cytoplasmic fractions of cell lysates using biotinylated anti-histone and peroxidase-coupled anti-DNA antibodies. The amount of nucleosomes is photometrically quantified at 405 nm by the peroxidase activity retained in the immunocomplexes. Apoptosis was defined as the specific enrichment of mono- and oligonucleosomes in the cytoplasm and was calculated by dividing the absorbance of treated samples by the absorbance of untreated samples after correcting for the number of cells. The induction of apoptosis in each melanoma cell line after a 7 h treatment with 2 μM staurosporin (100% apoptotic cells) was used to calculate the number of apoptotic cells.

### Comet Assay

DNA damage in cells was evaluated using the Single Cell Gel Electrophoresis Alkaline Assay from Trevigen® according to the manufacturer's instructions.

### Invasion Assay

Invasion assays were performed using a cell invasion assay kit (BD Bioscience). Melanoma cell suspensions were added in serum free medium and allowed to migrate for 48 h. The invading cells were stained and quantified measuring the surface occupied by stained cells with a cell counter plug-in of the Image J software.

### Reconstituted Skin

The melanoma skin model was obtained from MatTek Corp. (Ashland, Mass.). The melanoma skin cells in the model (containing A375 melanoma cells) were grown at the air/liquid interface and maintained in MCDB153 basal medium (MatTek Corp.), which was replenished every 2 days. Treatments were initiated seven days after tumor cells implantation.

### Mouse Melanoma Models

Animals were bred and maintained according to the Spanish legislation on the ‘Protection of Animals used for Experimental and other Scientific Purposes’ and in accordance with the directives of the European community. For subcutaneous melanoma model, B16/F10 cells (5.0×105) were subcutaneously injected into the dorsal flanks of 6-8 week-old female C57BL/6 mice. Animals with tumors greater than 8 mm in diameter on day 8 or with no visible tumor growth by day 12 were excluded. Groups (10 mice per group) were subjected to treatments starting at day 8 after tumor cell injection. Mice were treated intradermally with MTX (0.1 mg/kg/day) and/or TMECG (10 mg/kg/day) 5 times a week for 3 weeks. Hepatic metastases were produced by intrasplenic injection of 3.0×105 B16-F10-luc-G5 mouse melanoma cells (Caliper Life Sciences, Hopkinton, Mass.) as previously described (see Vidal-Vanaclocha, F. et al.). Primary spleen tumors and hepatic metastases at 12 and 14 days, respectively, were analyzed using the IVIS Imaging System (Caliper Life Sciences). In order to study the effect of the MTX/TMECG combination on hepatic metastases, mice were treated intraperitoneally with MTX (1 mg/kg/day) and/or TMECG (10 or 50 mg/kg/day) from day 1 to 14. Control mice received the same volume of vehicle (DMSO). To confirm the presence of melanoma cells in the livers of mice, a post-mortem with histological examination of the livers was performed in all animals. Tissues were fixed in 10% formaldehyde, dehydrated and embedded in paraffin wax. Sections (4 μm) were stained with hematoxylin and eosin (H&E). A Leica DMRB microscope connected to a Leica DC500 digital camera was used to quantify the number, average diameter, and position coordinates of metastases. Percentage of liver volume occupied by metastases was also determined (Vidal-Vanaclocha).

A minimum of five sections stained with H&E from each tumor were selected for evaluating the extent of necrosis, which was quantified: percentage necrosis was analyzed with the image-analysis program Image-Pro plus (Media Cybernetics, Silver Spring, Md.). For TYR detection in mouse livers, livers (3 per treatment) were cut into approximately 0.2 g slices. Five randomly chosen slices from each liver were used for phenol-chloroform total RNA extraction. RNA (5 μg) was then used to synthesize cDNA, and equal amounts of the five cDNA fractions corresponded to the same liver were pooled and employed for TYR mRNA determinations using realtime RT-PCR (TYR primers: forward: 5′-GGG CCC AAA TTG TAC AGA GA-3′; reverse: 5′-ATG GGT GTT GAC CCA TTG TT-3′).

### PCR Analysis

mRNA extraction, cDNA synthesis, and conventional and qRT-PCR were performed under standard conditions. Primers were designed using Primer Express version 2.0 software (Applied Biosystems, Foster City, Calif.) and synthesized by Invitrogen (Barcelona, Spain). The following primers for human genes were used: β-Actin (forward: 5′-AGA AAA TCT GGC ACC ACA CC-3′; reverse: 5′-GGG GTG TTG AAG GTC TCA AA-3′), Apaf1 (forward: 5′-GCT CTC CAA ATT GAA AGG TGA AC-3′; reverse: 5′-ACT GAA ACC CAA TGC ACT CC-3′), MART1 (forward: 5′-TGG ATA AAA GTC TTC ATG TTG GC-3′; reverse: 5′-GTG GAG CAT TGG GAA CCA C-3′), MITF (forward: 5′-GCG CAA AAG AAC TTG AAA AC-3′; reverse: 5′-CGT GGA TGG AAT AAG GGA AA-3′), p.53 (forward: 5′-TAA CAG TTC CTG CAT GGG CGG C-3′; reverse: 5′-AGG ACA GGC ACA AAC ACG CAC C-3′), Pmel17 (forward: 5′-AAG GTC CAG ATG CCA GCT CAA TCA-3′; reverse: 5′-AGG ATC TCG GCA CTT TCA ATA CCC-3′), TAp73 (forward: 5′-TGG AAC CAG ACA GCA CCT ACT TCG-3′; reverse: 5′-CAG GTG GCT GAC TTG GCC GTG CTG-3′), Rab27a (forward: 5′-GCC ACT GGC AGA GGC CAG-3′; reverse: 5′-GAG TGC TAT GGC TTC CTC CT-3′), TYR (forward: 5′-TTG GCA GAT TGT CTG TAG CC-3′; reverse: 5′-AGG CAT TGT GCA TGC TGC TT-3′), and TYRP1 (forward: 5′-GAT GGC AGA GAT GAT CGG GA-3′; reverse: 5′-AGA AAT TGC CGT TGC AGT GAC-3′).

### Stealth RNA Transfection

Specific Stealth siRNAs for MITF (HSS142939 and HSS142940) and p53 (HSS129934 and HSS129936) were obtained from Invitrogen and transfected into melanoma cells using Lipofectamine 2000 (Invitrogen). Treatments were started 24 h after siRNA transfection. Stealth RNA negative control duplexes (Invitrogen) were used as control oligonucleotides, expression of the selected genes was analyzed by western blotting 24 h after siRNA transfection.

### ChIP Assays

The ChIP assay was performed with the Magna ChIP™ G kit from Millipore according to the manufacturer's instructions. Briefly, untreated and MTX-treated SK-MEL-28 melanoma cells were formaldehyde cross-linked, and the DNA was sheared by sonication to give an average size of 300 to 3,000 bp. The cross-linked chromatin was then used for immunoprecipitation with MITF antibody, HDAC3 antibody (positive control) or mouse IgG (negative control). DNA from lysates prior to immunoprecipitation was used as positive input controls. After washing, elution, and DNA purification, the DNA solution (2 μL) was used as a template for qRT-PCR amplification using specific human primers: Pmel17 (forward: 5′-CAT AAG ATA CCC CAT TCT TTC TCC ACT T-3′; reverse: 5′-GAG AAT GTG GTA TTG GGT AAG AAC AC-3′); TYR (forward: 5′-GCT CTA TTC CTG ACA CTA CCT CTC-3′; reverse 5′-CAA GGT CTG CAG GAA CTG GCT AAT TG-3′) and GAPDH (forward: 5′-CAA TTC CCC ATC TCA GTC GT-3′; reverse: 5′-TAG TAG CCG GGC CCT ACT TT-3′).

Negative control regions (CR) for Pmel17 (forward: 5′-CAT GGA GAA CTT CCA AAA GGT GG-3′; reverse: 5′-TAC TCT CCC CAG GGA GTA TAA GT-3′) and TYR (forward: 5′-CAA TAT GGC TAC AGC ATT GGA G-3′; reverse: 5′-TCT CTC CCC TCT ATC CTC TCT CT-3′) were also used for PCR amplification. Standard curves were generated for all primer set to confirm linearity of signals over the experimentally measured ranges.

### Immunoblotting and Immunoprecipitation

Whole cell lysates were collected by adding SDS sample buffer. After extensive sonication, samples were boiled for 10 min and subjected to SDS-PAGE. Proteins were then transferred to nitrocellulose membranes and analyzed by immunoblotting (ECL Plus, GE Healthcare, Barcelona, Spain). Primary splenic tumors were cleaned, washed twice in PBS, and immediately frozen at −80° C. After thawing, tumors were chopped into 0.2-0.4-cm pieces, in order to increase the exposed surface, and then homogenizer in buffer [10 mM PBS pH 7.4, 1% NP-40, 150 mM NaCl, 0.5% sodium deoxycholate, 0.1% SDS (w/v), and protease inhibitor cocktail] using polytron and Potter homogenizers.

For immunoprecipitation assays, cells (˜5×10⁶) were lysed in 500 μL of lysis buffer (50 mM Tris, pH 8.0, 300 mM NaCl, 0.4% NP40, 10 mM MgCl₂) supplemented with protease and phosphatase inhibitor cocktails (Sigma). Cell extracts were cleared by centrifugation (20,000 g for 15 min) and then diluted with 500 μl of dilution buffer (50 mM Tris, pH 8.0, 0.4% NP40, 2.5 mM CaCl₂) supplemented with protease and phosphatase inhibitor cocktails and DNase I (Sigma). Extracts were pre-cleared by 30 min incubations with 20 μL of PureProteome Protein G Magnetic Beads (Millipore) at 4° C. with rotation. The antibodies (as indicated in the Figure. legends) were then added to the pre-cleared extracts. After incubation for 1 h at 4° C., 50 μL of PureProteome Protein G Magnetic Beads were added, and the extracts were further incubated for 20 min at 4° C. with rotation. After extensive washing, bound proteins were analyzed by western blotting. Unbound extracts were used as positive inputs for protein load determination.

dNTP Pool Extraction and Analysis

Asynchronously proliferating SK-MEL-28 cells were seeded in six-well dishes. The extraction and analysis of the dNTP pools in each extract were carried out as described previously (Angus, S. P. et al.). The reaction mixtures (50 μl) contained 100 mM HEPES buffer, pH 7.5, 10 mM MgCl₂, 0.1 units of the Escherichia coli DNA polymerase I Klenow fragment (Sigma, Madrid, Spain), 0.25 μM oligonucleotide template, and 1 μCi [³H]dATP (ARC, St. Louis, Mo.) or [³H]dTTP (Perkin-Elmer, Waltham, Mass.). Incubation was carried out for 60 min at 37° C.

### Microscopy

For scanning electron microscopy MTX-treated and untreated SK-MEL-28 cells were processed as described elsewhere (Serafino) and examined on a JEOL-6100 scanning electron microscope (Tokyo, Japan). Confocal microscopy was carried out using a Leica TCS 4D confocal microscope (Wetzlar, Germany). For indirect immuno-fluorescence studies, preparation of the cells on glass slides were fixed with cold acetone for 5 min, and washed with PBS. The cells were incubated with 3% bovine serum albumin (BSA) for 20 min and then 2 h at room temperature with primary antibodies (diluted 1:200 in PBS containing 1% BSA) as described in each experiment. The cells were washed three times in PBS and incubated for 1 h at room temperature with Alexa Fluor Dyes (Invitrogen) as secondary antibodies. After 3 washes with PBS, the cells were incubated with 0.01% 4′-6-diamidino-2-phenylidene (DAPI; Sigma) in water for 5 min. For antibody specificity, primary antibodies were replaced with specific IgGs (diluted 1:200) during immunofluorescence.

Positive γH2AX foci cells were evaluated in at least 10 fields at 960× magnification and γH2AX foci number was measured in at least twenty cells at 3,500× magnification. To improve signal to noise ratio, images were processed with the Huygens deconvolution package (SVI, Netherlands). A negative control (i.e. cells exposed to a nonspecific mouse IgG1) was run alongside each anti-γH2AX monoclonal antibody exposed group. For ionizing radiation assays (IR) cells were irradiated with an Andrex SMART 200E machine (YXLON International, Hamburg, Germany) operating at 200 kV, 4.5 mA, focus-object distance 20 cm at room temperature and at dose rate of 2.5 Gy per min. The radiation doses were monitored by a UNIDOS® universal dosimeter with PTW Farme® ionization chamber TW 30010 (PTW-Freiburg, Freiburg, Germany) in the radiation cabin.

### Reconstituted Skin

The melanoma skin model was obtained from MatTek Corp. (Ashland, Mass.). Cultures were prepared by plating single-cell suspensions of normal human epidermal keratinocytes and A375 melanoma cells at a 1:10 ratio on fibroblast-contracted collagen gels within cell culture inserts. They were allowed to grow and differentiate in DMEM-based serum-free medium, forming three-dimensional, highly differentiated, full-thickness, skin-like tissues. On day 7, culture inserts were incubated in duplicate with MCDB153 basal medium (MatTek Corp.) containing DMSO (vehicle), MTX and/or TMECG. Medium, containing the indicated active agents was replenished every other day, and cultures collected on days 12, 17, and 21 and fixed with 10% formalin. Culture inserts were paraffin embedded, sectioned, and analyzed by H&E staining. A375 cell infiltration was evaluated at a 120× magnification using Imagej v1.45s (rsbweb.nih.gov/ij/) software. Ten different microscopic sections, for each treatment, were used to compare tumor area versus skin area. The area was normalized to the skin within the field of observation, which area was also calculated. All skin pieces were also outlined manually with the image analysis software to be 440 μm thick so the ratios can be compared.

### Primary Melanoma Cells and Lentivirus Infection

Tumor samples were collected from patients attending the melanoma service at Oxford University Hospital, all of whom provided written informed consent. The protocol was approved by Oxfordshire Research Ethics Committee C (reference 09/H0606/5). Human melanoma biopsies were minced using a rotary cutter and further dissociated with Dispase II and Collagenase P (3 mg/mL each, Roche) in RPMI1640 medium containing 10% FCS for 1 h at 37° C. The cells were strained through a 100 μm filter, centrifuged and washed in complete medium until the supernatant was clear. Recombinant lentivirus was made in Phoenix producer cells using the lentiviral vector pCSII-EF-MCS (a kind gift of H. Miyoshi), in which the EF1a promoter was replaced with a 1 kb fragment of the human DCT promoter, driving mCherry expression only in the melanocyte lineage. Phoenix cells were seeded onto poly-L-Lysine coated plates and transfected with pCSII-pTRP-mCherry and Gag/Pol/Rev and VSV containing vectors. Medium was changed after 24 h and the virus-containing supernatant was harvested after 48 h and 72 h after transfection. After filtration through a 0.45 μm syringe filter viral particles were concentrated by centrifugation at 50,000 g for 2 hr and resuspended in Hepes buffered saline containing 1 mg/ml polybrene. Primary human cells were infected by removal of culture medium and incubation with the concentrated viral suspension for 5 min. Fresh medium was added and replaced after 24 hr. Viral infection typically achieved an efficiency of >80% of primary cells.

### Pharmacokinetic Studies

Pharmacokinetic studies were performed in male C57BL/6 mice after intra-peritoneal injection of MTX (50 mg/kg) and/or TMECG (50 mg/kg). Animals were anesthetized with CO₂, and whole blood samples (˜0.5 mL) were collected via cardiac puncture. Samples for drug quantification were collected from different animals (in triplicate) at 5, 15, and 30 min and at 1, 2, 4, and 6 hr post-dose. Whole blood samples were transferred to lithium heparin blood collection tubes, and then centrifuged at 10,000 rpm for 10 min at 4° C. After plasma deproteinization with 5% trichloroacetic acid (TCA), MTX and TMECG plasma concentrations were calculated by HPLC. Pharmacokinetic parameters were estimated using the WinNonlin software package (WinNonlin Professional version 5.1., Pharsight Corporation, CA). WinNonlin model 200 was used for the non-compartmental analysis of the concentration-time data. The area under the plasma concentration-time curve (AUCₗₐₛₜ) from time 0 to the last point (tₗₐₛₜ) with measurable concentration (Cₗₐₛₜ) was estimated using a linear trapezoidal approximation (AUCₗₐₛₜ). The elimination half-life (t_{1/2}) was calculated as ln2/kₑₗ, where kₑₗ (elimination rate constant) was estimated using least squares regression analysis of the concentration-time data obtained during the terminal log-linear elimination phase. The maximum plasma concentrations (Cₘₐₓ) were estimated directly from the data, with tₘₐₓ being defined as the time of the first occurrence of Cₘₐₓ.

### Toxicology

To explore the toxicity of TMECG and MTX, these agents (at indicated concentrations) were administrated intradermally to the back of non-tumor inoculated female C57BL/6 mice (n=10), and body weight monitored every other day. For microscopic analysis of mice eyes, animals were perfused transcardially with 4% paraformaldehyde (PFA) in phosphate buffer 0.1 M after a saline rinse. The eyes were enucleated and the superior pole of the sclera marked with a suture and the superior rectus muscle used to maintain their orientation. The whole eyes were postfixed for 24 h in the same fixative, dehydrated through alcohols and 1-butanol, and embedded in paraffin. Five-micron-thick cross sections were obtained in a rotational microtome (Microm HM-340-E; Microm Laborgerate GmbH, Walldorf, Germany) and mounted on slides coated with 0.01% poly-L-lysine (Sigma). A series of sections were deparaffinized, rehydrated, stained with Hansen's H&E, and mounted with DePex (BDH Laboratory Supplies, Poole, UK) (Montalbán-Soler et al., 2012). The integrity of RPE and iris was studied. The number of skin melanocytes was determined by immunohistochemistry and immunofluorescence using MART1 and MITF antibodies, respectively. Positive cells for each antibody in hair follicles (minimum of 3 sections per mouse) were counted and expressed relative to the number of positive cells in untreated mice. For immunohistochemistry paraffin-embedded tissue sections were deparaffinized, rehydrated in PBS, and treated with proteinase K for 5 min at 37° C. for antigen retrieval. Appropriate positive and negative controls were included for each antibody test.

### MALDI-TOF Mass Spectroscopy

SK-MEL-28 whole cell lysates were immunoprecipitated as described above but with two variations. First, the lysis and dilution buffers contained 2.5 μM trichostatin (a potent deacetylase inhibitor) and 20 μM trans-2-phenylcyclo-propylamine (an irreversible inhibitor of lysine-specific demethylase 1, LSD1). Second, the E2F1 antibody was covalently coupled to Dynabeads® (Invitrogen). After immunoprecipitation and elution, bound proteins were digested with trypsin according to standard procedures (Shevchenko). Data were recorded and processed with Agilent MassHunter Workstation Software for obtaining the Peptide Mass Fingerprint (PMF). The PMF result mass spectra were searched against the E2F1 protein sequence with carbamidomethylation of cysteine as fixed modification and methylation and acetylation of lysine residues, oxidation of methionine residues and phosphorylation of serine residues as variable modifications. Peptide mass tolerance was set to 50 ppm and a maximum of three missed cleavages was considered.

### Image Acquisition, Quantification of Western Blots, and Statistical Analysis

Western blot and microscopy data have been repeated at least three times, and similar results were obtained. The results from one experiment are shown. For quantification, western blot results were scanned with a Bio-Rad ChemiDoc scanning densitometer (Bio-Rad Laboratories, Hercules, Calif.). For other experiments, the mean±SD for 5 determinations in triplicate were calculated. Numeric data were analyzed for statistical significance using Mann-Whitney test for comparison of means with SPPS statistical software for Microsoft Windows, release 6.0 (Professional Statistic, Chicago, Ill.). Individual comparisons were made with Student's two-tailed, unpaired t test. The criterion for significance was P<0.05 for all comparisons.

TABLE 1
HPLC/MS analyses of MTX, TMECG-QM, and DHF in whole cell
extracts of SK-MEL-28 melanoma cells subjected to 24 h MTX (1 μM) and
TMECG (10 μM) individual and combined treatments

| | | TMECG-QM** *** | |
|---|---|---|---|
| Treatment | MTX | (pmol/10⁶ cells) | DHF** *** |
| Control | n.d. | n.d. | 3.8 ± 1.1 |
| MTX | <0.001* | n.d. | 0.21 ± 0.05 |
| TMECG | n.d. | 41 ± 9 | 4.8 ± 0.9 |
| MTX/TMECG | <0.001* | 247 ± 30 | 0.18 ± 0.05 |

n.d. non determined.
*Intracellular concentrations of MTX were determined by HPLC/MS/MS as described previously (see Guo P et al.). Low values of MTX in melanoma cells may be related with cellular exportation of the drug. Using the same methodology, intracellular levels of MTX in MCF7 and Caco-2 subjected to the same MTX treatment were calculated to be 1.3 ± 0.4 and 0.8 ± 0.3 pmol/106 cells, respectively. The concentration of MTX in SK-MEL-28 was not affected by the presence of TMECG in the treatment, which indicated that this compound did not interfered with the mechanisms responsible of the melanosome sequestration and exportation of MTX in melanoma cells.
**Intracellular concentrations of TMECG-QM and DHF were determined by HPLC/MS/MS as described elsewhere (Sáez-Ayala et al., 2011).
***Although TMECG-QM inhibited DHFR with an inhibition constant in the nanomolar order of concentration (Sanchez-del-Campo et al., 2009a), it does not inhibit other EGCG-proposed targets such as 5-cytosine DNA methyltransferase-1 (Fang et al., 2003), glutamate dehydrogenase (Li et al., 2006), or the proteasome (Nam et al., 2001) (data not shown). The higher concentration of TMECG-QM in MTX/TMECG-treated cells together with depressed folate level by MTX may favor TMECG-QM competitive inhibition of DHFR in cells treated with the combination MTX/TMECG.

TABLE 2
MALDI-TOF mass spectroscopy properties of immunoprecipitated
E2F1 tryptic digests

| | | | | | | MTX^{b} | |
|---|---|---|---|---|---|---|---|
| | | | Measured^{a} | Theoretical^{a} | | Inten- | MTX/ |
| Modification | E2F1 Status | Peptide Sequence^{a} | (m/z) | (m/z) | CN^{b} | sity^{c} | TMECG^{b} |
| Methylation | Non-methylated | (K)NHIQWLGSHTTVGVGGR(L) | 1820.0299 | 1820.0331 | 64 | 379 | 526 |
| (K185) | Methylated | (K)SK_{Me}NHIQWLGSHTTVGVGGR(L) | 2049.3135 | 2049.3140 | 355 | 18 | 12 |
| Acetylation | Non-acetylated | (R)HPGK(G) | 438.5101 | 438.5097 | 325 | 16 | 21 |
| (K117,120,125) | | (K)SPGEK(S) | 517.5611 | 517.5625 | 320 | 20 | 18 |
| | Hyperacetylated | (R)HPGK_{Ac}GVK_{Ac}SPGEK_{Ac}SR(Y) | 1589.8399 | 1589.8394 | 49 | 329 | 493 |
| Phosphorylation | Non- | (R)LLDSSQIVIISAAQDASAPPAPTGP | 6275.2592 | 6275.2600 | 295 | 312 | 16 |
| (S31) | phosphorylated | AAPAAGPC(Carbamidomethyl)DPDLLL | | | | | |
| | | FATPQAPRPTPSAPRPALGRPPVK(R) | | | | | |
| | Phosphorylated | (R)LLDSS_{P}QIVIISAAQDASAPPAPTGP | 6355.2361 | 6355.2398 | 46 | 12 | 456 |
| | | AAPAAGPC(Carbamidomethyl)DPDLLL | | | | | |
| | | FATPQAPRPTPSAPRPALGRPPVK(R) | | | | | |
| Phosphorylation | Non- | (R)MGSLR(A) | 563.7085 | 563.7006 | 318 | 310 | 20 |
| (S364) | phosphorylated | | | | | | |
| | Phosphorylated | (R)MGS_{P}LR(A) | 643.6799 | 643.6804 | 11 | 24 | 519 |

^{a}The characteristics peptides involving posttranslational modifications of E2F1 (methylation = Me, acetylation = Ac, and phosphorylation = P), as well as their measured and theoretical m/z are shown;
^{b}Peptides were analyzed in untreated SK-MEL-28 cells (CN), treated for 24 h with 1 μM MTX (MTX) or treated for 24 h with 1 μM MTX plus 10 μM TMECG (MTX/TMECG);
^{c}Relative intensities of specific tryptic peptides were normalized with respect to an internal matrix control.

TABLE 3
MTX and TMECG pharmacokinetic parameters in mice plasma

| | MTX parameters | | | | TMECG Parameters | | | |
|---|---|---|---|---|---|---|---|---|
| | Cₘₐₓ | Tₘₐₓ | AUC | t_{1/2} | Cₘₐₓ | Tₘₐₓ | AUC | t_{1/2} |
| Treatment | (mg/L) | (h) | (mg·h/L) | (h) | (mg/L) | (h) | (mg·h/L) | (h) |
| MTX | 24.60 | 0.5 | 32.03 | 1.77 | | | | |
| TMECG | | | | | 39.26 | 0.09 | 79.10 | 1.79 |
| MTX/TMECG | 23.63 | 0.5 | 31.18 | 1.65 | 40.74 | 0.09 | 85.82 | 2.03 |

### REFERENCES

All documents mentioned in this specification are incorporated herein by reference in their entirety.
Angus, S. P. et al. J. Biol. Chem. 277, 44376-44384 (2002)
Arnheiter, H. Pigment Cell Melanoma Res 23, 729-735 (2010).
Ascierto, P. A. et al. J Transl Med 8, 38 (2010).
Balch C. et al. J. Clin. Oncol. 19, 3635-48 (2001).
Bardeesy, N. et al Mol. Cell. Biol. 25, 4176 (2005).
Bertolotto, C. et al. Nature 480, 94-98 (2011).
Blagosklonny, M. V. Cell Cycle 4, 1693-1698 (2005).
Boni, A. et. al Cancer Research 70, 5213-5219 (2010).
Box, N. F. and Terzian, T. Pigment Cell Melanoma Res 21, 525-533 (2008).
Carreira, S. et al., Nature 433, 764-769. (2005).
Carreira, S. et al., Genes Dev 20, 3426-3439 (2006).
Chapman, P. B. et al. J. Clin. Oncol. 17, 2745 (1999)
Chapman, P. B. et al. New. Eng. J. Med. 364, 2507-2516 (2011)
Cheli, Y. et al., Oncogene 31, 2461-2470 (2011).
Cheli, Y. et al., Oncogene 30, 2307-2318 (2011).
Cheli, Y., et al. Pigment Cell Melanoma Res 23, 27-40 (2010)
Chin, L. et al. Genes Dev. 20, 2149 (2006)
Davies, H. et al., Nature 417, 949-954. (2002)
Dobbelstein, M., et al. Biochem Biophys Res Commun 331, 688-693 (2005)
Fang et al. Cancer Res 63, 7563-7570 (2003)
Garraway, L. A. et al. Nature 436, 117-122 (2005)
Guo P, et al. J Pharm Biomed Anal 2007; 43: 1789-1795.
Hoek, K. S. and Goding, C. R., Pigment Cell Melanoma Res 23, 746-759 (2010)
Jordan et al. Bioorg. Med. Chem. 1999, 7, 1775-1780
Jordan et al. Bioorg. Med. Chem. 2001, 9, 1549-1558
Kinner, A. et al. Nucleic Acids Res. 36, 5678-5694 (2008)
Kontaki, H. and Talianidis, I. Mol Cell 39, 152-160 (2010)
Lee M. et al. J. Biol Chem 275, 37978-37983 (2000)
Loercher, A. E., et al. J. Cell. Biol. 168, 35-40 (2005)
Lopez-Bergami, P. Pigment Cell Melanoma Res 24, 902-921 (2011)
Martinez-Balbas, M. A. et al. EMBO J 19, 662-671 (2000)
Montalbán-Soler et al. Mol Vis 18, 675-93 (2012)
Nam et al. J Biol. Chem. 276, 13322-13330 (2001)
Nazarian, R. et al. Nature 468, 973-977 (2010)
Nikolaev et al. Nature Genetics 44, 133-139 (2011)
Pardee, A. B et al. Cell Cycle 3, 1091-1094 (2004)
Patel, P. M. et al. Eur. J. Cancer 47, 1476 (2011)
Poulikakos, P. I. Nature 480, 387-390 (2011)
Rhodes, A. R. et al. JAMA. 258, 3146 (1987)
Sáez-Ayala et al. Chem Med Chem 6, 440-449 (2011)
Sanchez-del-Campo, L. et al. J. Med. Chem. 51, 2018-2026 (2008)
Sánchez-del-Campo, L. and Rodriguez-López, J. N. Int J Cancer 123, 2446-2455 (2008).
Sanchez-del-Campo, L. et al. Mol Pharm 6, 883-894 (2009).
Sanchez-del-Campo, L., et al. Melanoma Res 22, 588-600 (2009).
Serafino, A. et al. FASEB J 18, 1940-1942 (2004).
Serone, L. et al. J. Exp. Clin. Cancer Res. 19, 21-34 (2000)
Shevchenko, A., Wilm, M., Vorm, O., and Mann, M. Anal Chem 68, 850-858 (1996).
Sosman, J. A. et al. N Engl J Med 366, 707-714 (2012).
Strub, T. et al. Oncogene 30, 2319-2332 (2011).
Sullivan, R. J. et al Expert Review of Anticancer Therapy 9, 567-581 (2009).
Tachibana et al. Nature Genetics 14, 50-54 (1996)
Tawbi, H. A. et al Clinical Advances in Hematology & Oncology, 8, 259-266 (2010)
Urist, M., et al. Genes Dev 18, 3041-3054 (2004).
Vidal-Vanaclocha, F. et al. Cancer Res 54, 2667-2672 (1994).
Villanueva, J. et al. Cancer Cell 18, 683-695 (2010)
Villanueva, et al. Cancer Res 71, 7137-7140 (2011).
Visvader, J. E. and Lindeman, G. J. Nat Rev Cancer 8, 755-768 (2008).
Wang, A., et al. Cancer Res 65, 7809-7814 (2005).
WO 2009/081275
Yokoyama, S. et al. Nature 480, 99-103 (2011).
Zaidi, M. R. et al. Nature 469, 548-553 (2011).

The invention is further described by the following numbered paragraphs:

1. A method of treatment of melanoma comprising:

administering a tyrosinase expression enhancer and a tyrosinase-activated prodrug to an individual in need thereof.

2. A tyrosinase expression enhancer for use in the treatment of melanoma in combination with a tyrosinase-activated prodrug.

3. A tyrosinase-activated prodrug for use in the treatment of melanoma in combination with a tyrosinase expression enhancer.

4. A combination of a tyrosinase expression enhancer and a tyrosinase-activated prodrug for use in the treatment of melanoma.

5. Use of a tyrosinase expression enhancer in the manufacture of a medicament for use in the treatment of melanoma in combination with a tyrosinase-activated prodrug.

6. Use of a tyrosinase-activated prodrug in the manufacture of a medicament for use in the treatment of melanoma in combination with a tyrosinase expression enhancer.

7. Use of a combination of a tyrosinase expression enhancer and a tyrosinase-activated prodrug in the manufacture of a medicament for use in the treatment of melanoma.

8. A pharmaceutical formulation comprising a tyrosinase expression enhancer and a tyrosinase-activated prodrug, optionally for use in the treatment of melanoma.

9. The pharmaceutical formulation according to paragraph 8 comprising a pharmaceutically acceptable carrier and optionally one or more additional active compounds.

10. A method, use, inhibitor, compound or formulation according to any one of paragraphs 1 to 9 wherein the tyrosinase-activated prodrug is for the treatment of melanoma.

11. A method, use, inhibitor, compound or formulation according to any one of paragraphs 1 to 9, wherein the tyrosinase-activated prodrug is a catechin compound.

12. A method, use, inhibitor, compound or formulation according to paragraph 11, wherein the catechin compound is a compound of formula (XI):

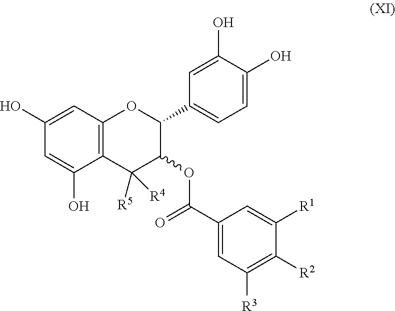

wherein:

each —R¹, —R² and —R³ is independently -Q¹, —OH or —H, where at least one of —R¹, —R² and —R³ is not —H or —OH;

each —R⁴ and —R⁵ is independently -Q² or —H;

each -Q¹ is independently selected from:

—F, —Cl,

—R^{A},

—OR^{A},

—SH, —SR^{A},

where each —R^{A} is independently selected from methyl and ethyl, which may substituted by one or more fluoro or chloro groups;

each -Q² is selected from:

—F, —Cl,

—R^{B},

—OR^{B},

—SH, —SR^{B},

where each —R^{B} is independently selected from methyl and ethyl, which may substituted by one or more fluoro or chloro groups or an isomer, salt, solvate or prodrug thereof.

13. A method, use, inhibitor, compound or formulation according to paragraph 12, wherein the catechin compound is TMECG or TMCG.

14. A method, use, inhibitor, compound or formulation according to paragraph 13, wherein the catechin compound is TMECG.

15. A method, use, inhibitor, compound or formulation according to any one of the preceding paragraphs, wherein the tyrosinase expression enhancer is a MITF expression enhancer.

16. A method, use, inhibitor, compound or formulation according to any one of the preceding paragraphs, wherein the tyrosinase expression enhancer is a DHFR inhibitor.

17. A method, use, inhibitor, compound or formulation according to paragraph 16, wherein the DHFR inhibitor reduces DHF levels in a cell.

18. A method, use, inhibitor, compound or formulation according to any one of the preceding paragraphs, wherein the tyrosinase expression enhancer is methotrexate (MTX).

19. A method of screening for a compound with activity in increasing tyrosinase levels in a cell, the method comprising:

contacting a cancer cell with a tyrosinase-activated prodrug and a test compound and determining the conversion of the prodrug to its active form,

wherein in increase in the conversion of the prodrug to its active form relative to the absence of test compound is indicative that the compound is active in increasing tyrosinase levels in a cell.

20. The method of paragraph 19, wherein the tyrosinase-activated prodrug is a catechin compound.

21. The method of paragraph 20, wherein the catechin compound is a compound of formula (XI), as defined in paragraph 12.

22. The method of paragraph 21, wherein the catechin compound is TMECG or TMCG.

23. The method of paragraph 22, wherein the catechin compound is TMECG.

24. The method of any of paragraphs 19 to 23, wherein the test compound has activity in increasing MITF expression in a cell.

25. The method of any of paragraphs 19 to 23, wherein the test compound is an antifolate compound.

26. The method of paragraph 25, wherein the antifolate compound reduces DHF levels in a cell.

27. A method of treatment of melanoma comprising:

administering a tyrosinase-activated prodrug and a compound for differentiating a stem-like tumor cell into a matured cell that is a tyrosinase producer to an individual in need thereof.

28. The method of paragraph 27, wherein the differentiation is associated with an increase in MITF levels in the cell.

29. The method of paragraph 27 or paragraph 28, wherein the tyrosinase-activated prodrug is a catechin compound.

30. The method of paragraph 29, wherein the catechin compound is TMECG or TMCG.

31. The method of paragraph 30, wherein the catechin compound is TMECG.

32. The method of any one of paragraphs 27 to 31, wherein the compound for differentiating a stem-like tumor cell is MTX.

33. A method of treatment of melanoma comprising:

administering a tyrosinase expression enhancer and a tyrosinase-activated prodrug to an individual in need thereof, wherein the individual has a melanoma in which one or more of BRAF, NRAS, p53, GNAQ, EGFR, PDGFR, RAC or c-kit carries a mutation.

34. The method of paragraph 33, wherein the individual has a melanoma in which BRAF carries a mutation.

35. The method of paragraph 34, wherein the BRAF mutation is selected from V600E, R461I, I462S, G463E, G463V, G465A, G465E, G465V, G468A, G468E, N580S, E585K, D593V, F594L, G595R, L596V, T598I, V599D, V599E, V599K, V599R, K600E and A727V.

36. The method of paragraph 35, wherein the BRAF mutation is V600E.

37. The method of any one of paragraphs 33 to 36, wherein the individual has developed phenotypic resistance to chemotherapy.

38. The method of any one of paragraphs 33 to 37, wherein the tyrosinase-activated prodrug is a catechin compound.

39. The method of paragraph 38, wherein the catechin compound is TMECG or TMCG.

40. The method of paragraph 39, wherein the catechin compound is TMECG.

41. The method of any one of paragraphs 33 to 40, wherein the tyrosinase expression enhancer is MTX.

Having thus described in detail preferred embodiments of the present invention, it is to be understood that the invention defined by the above paragraphs is not to be limited to particular details set forth in the above description as many apparent variations thereof are possible without departing from the spirit or scope of the present invention.

What is claimed is:

1. A method of treatment of melanoma comprising:
administering a tyrosinase expression enhancer and a tyrosinase-activated prodrug to an individual in need thereof.

2. A pharmaceutical formulation comprising a tyrosinase expression enhancer and a tyrosinase-activated prodrug.

3. A method according to claim 1 wherein the tyrosinase-activated prodrug is for the treatment of melanoma.

4. A method according to claim 1, wherein the tyrosinase-activated prodrug is a catechin compound.

5. A method according to claim 4, wherein the catechin compound is a compound of formula (XI): 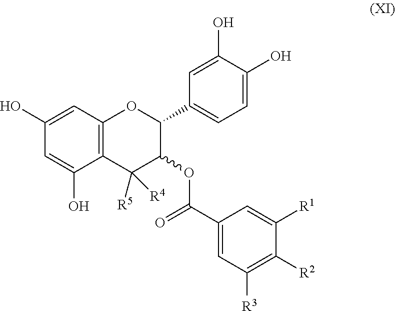
wherein:
each —R¹, —R² and —R³ is independently -Q¹, —OH or —H, where at least one of —R¹, —R² and —R³ is not —H or —OH;
each —R⁴ and —R⁵ is independently -Q² or —H;
each -Q¹ is independently selected from:
—F, —Cl,
—R^{A},
—OR^{A},
—SH, —SR^{A},
where each —R^{A} is independently selected from methyl and ethyl, which may substituted by one or more fluoro or chloro groups;
each -Q² is selected from:
—F, —Cl,
—R^{B},
—OR^{B},
—SH, —SR^{B},
where each —R^{B} is independently selected from methyl and ethyl, which may substituted by one or more fluoro or chloro groups
or an isomer, salt, solvate or prodrug thereof.

6. A method according to claim 5, wherein the catechin compound is TMECG or TMCG.

7. A method according to claim 1, wherein the tyrosinase expression enhancer is a MITF expression enhancer.

8. A method according to claim 7, wherein the tyrosinase expression enhancer is a DHFR inhibitor.

9. A method according to claim 8, wherein the DHFR inhibitor reduces DHF levels in a cell.

10. A method according to claim 1, wherein the tyrosinase expression enhancer is methotrexate (MTX).

11. A method of screening for a compound with activity in increasing tyrosinase levels in a cell, the method comprising:
contacting a cancer cell with a tyrosinase-activated prodrug and a test compound and determining the conversion of the prodrug to its active form,
wherein an increase in the conversion of the prodrug to its active form relative to the absence of test compound is indicative that the compound is active in increasing tyrosinase levels in a cell.

12. The method of claim 11, wherein the tyrosinase-activated prodrug is a catechin compound.

13. The method of claim 12, wherein the catechin compound is a compound of formula (XI): 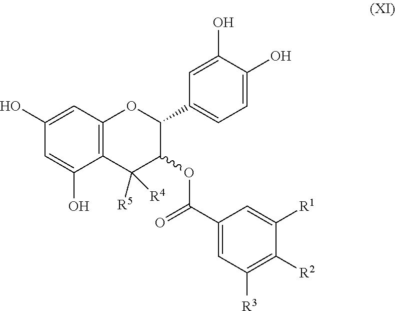
wherein:
each —R¹, —R² and —R³ is independently -Q¹, —OH or —H, where at least one of —R¹, —R² and —R³ is not —H or —OH;
each —R⁴ and —R⁵ is independently -Q² or —H;
each -Q¹ is independently selected from:
—F, —Cl,
—R^{A},
—OR^{A},
—SH, —SR^{A},
where each —R^{A} is independently selected from methyl and ethyl, which may substituted by one or more fluoro or chloro groups;
each -Q² is selected from:
—F, —Cl,
—R^{B},
—OR^{B},
—SH, —SR^{B},
where each —R^{B} is independently selected from methyl and ethyl, which may substituted by one or more fluoro or chloro groups
or an isomer, salt, solvate or prodrug thereof.

14. The method of claim 13, wherein the catechin compound is TMECG or TMCG.

15. The method of claim 11, wherein the test compound has activity in increasing MITF expression in a cell.

16. The method of claim 11, wherein the test compound is an antifolate compound.

17. A method of treatment of melanoma comprising:
administering a tyrosinase-activated prodrug and a compound for differentiating a stem-like tumor cell into a matured cell that is a tyrosinase producer to an individual in need thereof.

18. The method of claim 17, wherein the differentiation is associated with an increase in MITF levels in the cell.

19. The method of claim 17, wherein the tyrosinase-activated prodrug is a catechin compound.

20. The method of claim 19, wherein the catechin compound is TMECG or TMCG.

21. The method of claim 17, wherein the compound for differentiating a stem-like tumor cell is MTX.

22. A method of treatment of melanoma comprising:
administering a tyrosinase expression enhancer and a tyrosinase-activated prodrug to an individual in need thereof, wherein the individual has a melanoma in which one or more of BRAF, NRAS, p53, GNAQ, EGFR, PDGFR, RAC or c-kit carries a mutation.

23. The method of claim 22, wherein the individual has a melanoma in which BRAF carries a mutation.

24. The method of claim 22, wherein the individual has developed phenotypic resistance to chemotherapy.

25. The method of claim 22, wherein the tyrosinase-activated prodrug is a catechin compound.

26. The method of claim 25, wherein the catechin compound is TMECG or TMCG.

27. The method of claim 22, wherein the tyrosinase expression enhancer is MTX.

28. The pharmaceutical formulation according to claim 2, wherein the tyrosinase-activated prodrug is a catechin compound.

29. The pharmaceutical formulation according to claim 28, wherein the catechin compound is a compound of formula (XI): 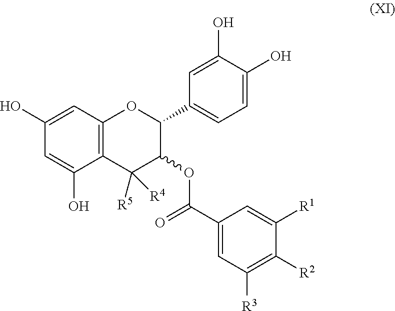
wherein:
each —R¹, —R² and —R³ is independently -Q¹, —OH or —H, where at least one of —R¹, —R² and —R³ is not —H or —OH;
each —R⁴ and —R⁵ is independently -Q² or —H;
each -Q¹ is independently selected from:
—F, —Cl,
—R^{A},
—OR^{A},
—SH, —SR^{A},
where each —R^{A} is independently selected from methyl and ethyl, which may substituted by one or more fluoro or chloro groups;
each -Q² is selected from:
—F, —Cl,
—R^{B},
—OR^{B},
—SH, —SR^{B},
where each —R^{B} is independently selected from methyl and ethyl, which may substituted by one or more fluoro or chloro groups
or an isomer, salt, solvate or prodrug thereof.

30. The pharmaceutical formulation according to claim 29, wherein the catechin compound is TMECG or TMCG.

31. The pharmaceutical formulation according to claim 28, wherein the tyrosinase expression enhancer is a MITF expression enhancer.

32. The pharmaceutical formulation to claim 28, wherein the tyrosinase expression enhancer is a DHFR inhibitor.

33. The pharmaceutical formulation according to claim 32, wherein the DHFR inhibitor reduces DHF levels in a cell.

34. The pharmaceutical formulation according to claim 28, wherein the tyrosinase expression enhancer is methotrexate (MTX).
